(19) United States
(12) Patent Application Publication
(10) Pub. No.: US 2021/0047327 A1
(43) Pub. Date: Feb. 18, 2021

(54) DIAGNOSTIC COMPOSITIONS FOR PET IMAGING, A METHOD FOR MANUFACTURING THE DIAGNOSTIC COMPOSITION AND ITS USE IN DIAGNOSTICS

(71) Applicants: AC IMMUNE SA, Lausanne (CH); LIFE MOLECULAR IMAGING SA, Matran (CH)

(72) Inventors: Johnny CASTILLO MELEAN, Berlin (DE); Thomas BETZEL, Berlin (DE); Mathias BERNDT, Berlin (DE); Hanno SCHIEFERSTEIN, Wiesbaden (DE); Heiko KROTH, Ecublens (CH); Jérôme MOLETTE, Prevessin Moens (FR); Vincent DARMENCY, Bougy-Villars (CH); Emanuele GABELLIERI, Lausanne (CH)

(21) Appl. No.: 16/964,969

(22) PCT Filed: Jan. 22, 2019
(86) PCT No.: PCT/EP2019/051497

§ 371 Date: Jul. 24, 2020

(30) Foreign Application Priority Data
Jan. 24, 2018 (EP) 18153327.4

(51) Int. Cl. C07D 471/14; A61K 51/04; C07B 59/00; A61K 47/12; A61K 47/02; A61K 47/10; G01N 33/60; G01N 33/68

(52) U.S. Cl. CPC C07D 471/14; A61K 51/0455; C07B 59/002; A61K 47/12; G01N 2800/2821; A61K 47/10; G01N 33/60; G01N 33/6896; A61K 47/02

(57) ABSTRACT

The present application relates to a diagnostic composition comprising:
a. a compound of Formula I,

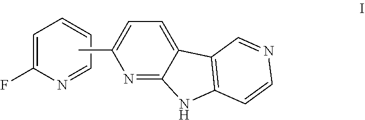

b. ethanol,
c. water, and
d. a hydroxycarboxylic acid, a salt of a hydroxycarboxylic acid or a mixture thereof.

The diagnostic composition can be used in the selective detection of disorders and abnormalities associated with Tau aggregates such as Alzheimer's disease (AD) and other tauopathies, for example, using Positron Emission Tomography (PET). The present invention also relates to a method of preparing the claimed diagnostic composition.

### FIELD OF INVENTION

The invention is directed to a diagnostic composition which is suitable for Positron Emission Tomography (PET) imaging. Further, the invention is directed to a method for manufacturing the diagnostic composition as well as the composition for use in diagnostics.

### BACKGROUND

Alzheimer's disease (AD) is a neurological disorder primarily thought to be caused by amyloid plaques, an extracellular accumulation of abnormal deposit of amyloid-beta (Aβ) aggregates in the brain or in the eyes. The other major neuropathological hallmarks in AD are the intracellular neurofibrillary tangles (NFT) that originate by the aggregation of the hyperphosphorylated Tau (Tubulin associated unit) protein, phosphorylated Tau or pathological Tau and its conformers. AD shares this pathology with many neurodegenerative tauopathies, in particularly with specified types of frontotemporal dementia (FTD). In AD brain, Tau pathology (tauopathy) develops later than amyloid pathology, but it is still discussed controversially if Aβ protein is the causative agent in AD which constitutes the essence of the so-called amyloid cascade hypothesis (Hardy et al., Science 1992, 256, 184-185, and most recently, Musiek et al., Nature Neurosciences 2015, 18(6), 800-806, “Three dimensions of the amyloid hypothesis: time, space and ‘wingmen’”).

Presently, the only definite way to diagnose AD is to identify plaques and tangles in brain tissue by histological analysis of biopsy or autopsy materials after the death of the individual. Beside AD, Tau plays an important role in other (non-AD) neurodegenerative diseases. Such non-AD tauopathies include, for example, supranuclear palsy (PSP), Pick's disease (PiD) and corticobasal degeneration (CBD).

The compound of general formula A has been proposed as being useful in the selective detection of disorders and abnormalities associated with Tau aggregates such as Alzheimer's disease (AD) and other tauopathies, and certain methods of manufacturing this compound have been described in the prior art.

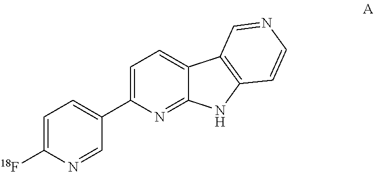

The pharmaceutical composition described in WO 2015/052105 and Gobbi et al. consists of [¹⁸F]-2-(6-fluoro-pyridin-3-yl)-9H-dipyrido[2,3-b;3′,4′-d]pyrrole in 1 mL ethanol and 10 mL saline. The components are passed through a 0.22 μm sterilizing filter.

¹⁸F-radiolabeled tracers for PET imaging are produced on demand and the diagnostic composition is usually used within 10 to 12 h after the end of manufacture. For long-distance shipment and for production of multiple doses out of one batch, the radioactivity level is increased (e.g. to achieve batches of [³F]fluorinated pyridinyl-9H-pyrrolo-dipyridines of ≥≥20 GBq or 50≥GBq or even ≥100 GBq.). Radiopharmaceuticals are known to be sensitive to radiolytic decomposition, which requires the use of stabilizing agents in suitable diagnostic compositions.

Especially for lipophilic compounds such as [¹⁸F]fluorinated pyridiny-9H-pyrroo-dipyridines loss on sterile filters and on surfaces (e.g. syringes) needs to be minimized for an efficient and reliable use of the diagnostic composition.

Therefore, it is an object of the present invention to provide a diagnostic composition which has improved stability.

### DESCRIPTION OF THE FIGURES

FIG. 1: Setup of the GE Traceriab FX synthesizer

FIG. 2: Setup of the IBA Synthera synthesizer

### SUMMARY OF THE INVENTION

The present invention relates to the following items:
1. A diagnostic composition comprising:
   a. a compound of Formula I,

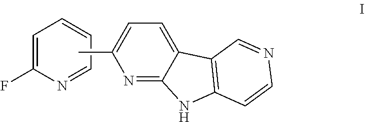

b. ethanol,
c. water, and
d. a hydroxycarboxylic acid, a salt of a hydroxycarboxylic acid or a mixture thereof.
2. A diagnostic composition according to item 1, wherein F in Formula I is ¹⁸F or ¹⁹F, preferably ¹⁸F or a mixture of ¹⁸F and ¹⁹F.
3. A diagnostic composition according to item 1 or 2, wherein the compound of Formula I is a compound of Formula Ib

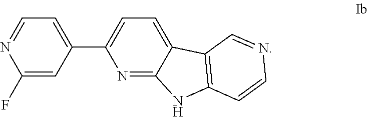 [SMILES: Fc1cc(-c2ccc3c(n2)[nH]c2ccncc23)ccn1]

4. A diagnostic composition according to any one of items 1 to 3 comprising about 0.03 GBq/mL to about 10 GBq/mL of the compound of Formula I, preferably about 0.03 GBq/mL to about 5 GBq/mL of the compound of Formula I.
5. A diagnostic composition according to any one of items 1 to 4 comprising at least about 1 GBq/mL of the compound of Formula I, preferably at least about 2 GBq/mL of the compound of Formula I, preferably at least about 3 GBq/mL of the compound of Formula I.
6. A diagnostic composition according to any one of items 1 to 5 comprising about 1% v/v to about 20% v/v ethanol, preferably about 1% v/v to about 15% v/v ethanol, more preferably about 5% v/v to about 10% v/v ethanol.
7. A diagnostic composition according to any one of items 1 to 6 wherein the hydroxycarboxylic acid, the salt of the hydroxycarboxylic acid or the mixture thereof are selected from the group consisting of ascorbic acid and salts of ascorbic acid, hydroxybenzoic acids and salts of hydroxybenzoic acids, hydroxybenzoic acid derivatives and salts of hydroxybenzoic acid derivatives, citric acid and salts of citric acid and mixtures thereof.
8. A diagnostic composition according to item 7, wherein the hydroxybenzoic acid derivative is selected from the group consisting of hydroxybenzoic acid, dihydroxybenzoic acid and trihydroxybenzoic acid.
9. A diagnostic composition according to item 8, wherein the dihydroxybenzoic acid is gentisic acid.
10. A diagnostic composition according to any one of items 1 to 9, wherein the hydroxycarboxylic acid, the salt of the hydroxycarboxylic acid or the mixture thereof is/are selected from ascorbic acid, sodium ascorbate, gentisic acid, gentisic acid sodium salt, citric acid, sodium citrate or a mixture thereof.
11. A diagnostic composition according to any one of items 1 to 10 comprising about 2.5 to about 500 μmol/mL of the hydroxycarboxylic acid, the salt of the hydroxycarboxylic acid or the mixture thereof, preferably about 10 to about 300 μmol/mL of the hydroxycarboxylic acid, the salt of the hydroxycarboxylic acid or the mixture thereof, more preferably about 25 to about 300 μmol/mL of the hydroxycarboxylic acid, the salt of the hydroxycarboxylic acid or the mixture thereof.
12. A diagnostic composition according to any one of items 1 to 7, 10 and 11, wherein the hydroxycarboxylic acid, the salt of the hydroxycarboxylic acid or the mixture thereof is/are selected from ascorbic acid, sodium ascorbate or mixture thereof, wherein the diagnostic composition preferably comprises about 10 to about 500 μmol/mL ascorbic acid, sodium ascorbate or mixture thereof, more preferably about 50 to about 500 μmol/mL ascorbic acid, sodium ascorbate or mixture thereof, even more preferably about 100 to about 500 μmol/mL ascorbic acid, sodium ascorbate or mixture thereof, even more preferably about 50 to about 300 μmol/mL ascorbic acid, sodium ascorbate or mixture thereof and still more preferably about 200 to about 300 μmol/mL ascorbic acid, sodium ascorbate or mixture thereof.
13. A diagnostic composition according to any one of items 1 to 11, wherein the hydroxycarboxylic acid, the salt of the hydroxycarboxylic acid or the mixture thereof is/are selected from gentisic acid, gentisic acid sodium salt or a mixture thereof, wherein the diagnostic composition preferably comprises about 2.5 to about 100 μmol/mL gentisic acid, gentisic acid sodium salt or a mixture thereof, more preferably about 10 to about 100 μmol/mL gentisic acid, gentisic acid sodium salt or a mixture thereof and even more preferably about 25 to about 75 μmol/mL gentisic acid, gentisic acid sodium salt or a mixture thereof.
14. A diagnostic composition according to any one of items 1 to 7, 10 and 11, wherein the hydroxycarboxylic acid, the salt of the hydroxycarboxylic acid or the mixture thereof is/are selected from citric acid, sodium citrate or a mixture thereof, wherein the diagnostic composition preferably comprises about 10 to about 500 μmol/mL citric acid, sodium citrate or a mixture thereof, more preferably about 50 to about 500 μmol/mL citric acid, sodium citrate or a mixture thereof and even more preferably about 50 to about 300 μmol/mL citric acid, sodium citrate or a mixture thereof.
15. A diagnostic composition according to any one of items 1 to 14 comprising an inorganic acid, an organic acid, a base, a salt or a mixture thereof, each of which is preferably diagnostically acceptable, wherein the organic acid, the salt or the mixture thereof is/are different from the hydroxycarboxylic acid, the salt of the hydroxycarboxylic acid or the mixture thereof.
16. A diagnostic composition according to item 15, wherein the inorganic acid, the organic acid, the base, the salt or the mixture thereof is/are selected from the group consisting of sodium chloride, potassium chloride, monosodium phosphate, disodium phosphate, trisodium phosphate, monopotassium phosphate, dipotassium phosphate, tripotassium phosphate, hydrochloric acid, phosphoric acid, sodium hydroxide and potassium hydroxide.
17. A diagnostic composition according to any one of items 1 to 16, wherein the pH of the diagnostic composition is about 4 to about 8.5.
18. A diagnostic composition according to any one of items 1 to 17 that is sterile.
19. A diagnostic composition according to any one of items 1 to 18 that is suitable for parenteral administration to a mammal.
20. A method for manufacturing a diagnostic composition as defined in any one of items 1 to 19 comprising the steps of:
   a. reacting a compound of Formula II with a ¹⁸F fluorinating agent,

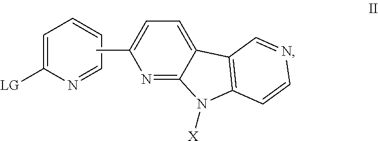

wherein X is H or PG,
LG is a leaving group, and
PG is an amine protecting group,
b. optionally, if X is PG, cleaving the protecting group PG,
c. purification of the compound of Formula I, and
d. optionally, mixing the compound of Formula I obtained in step c) with one or more selected from the group consisting of ethanol, water, the hydroxycarboxylic acid and the salt of the hydroxycarboxylic acid to provide the diagnostic composition.
21. The method for manufacturing a diagnostic composition according to claim 20, wherein one or more of an inorganic acid, an organic acid, a base, or a salt are additionally admixed in step d, wherein the organic acid, the salt or the mixture thereof is/are different from the hydroxycarboxylic acid, the salt of the hydroxycarboxylic acid or the mixture thereof.
22. A method according to item 20 or 21, further comprising
   e. sterile filtration before or after step d).
23. A method according to any one of items 20 to 22, wherein LG in Formula II is a leaving group, which can be substituted by a nucleophilic [¹⁸F]fluoride ion or an electrophilic [¹⁸F]fluorine atom, preferably LG is selected from the group consisting of nitro, bromo, iodo, chloro, trialkyl ammonium, hydroxyl, boronic acid, iodonium, sulfonic ester, more preferably LG is nitro or trimethyl ammonium, wherein the compounds containing trialkyl ammonium or iodonium may further comprise an anion.
24. A method according to any one of items 20 to 23, wherein PG in Formula is a protecting group, preferably PG is selected from the group consisting of carbobenzyloxy (Cbz), (p-methoxybenzyl)oxycarbonyl (Moz or MeOZ), tert-butyloxycarbonyl (BOC), 9-fluorenylmethyloxycarbonyl (FMOC), benzyl (Bn), p-methoxybenzyl (PMB), 3,4-dimethoxybenzyl (DMPM), p-methoxyphenyl (PMP), triphenylmethyl (Trityl), methoxyphenyl diphenylmethyl (MMT), or dimethoxytrityl (DMT), more preferably PG is selected from tert-butyloxycarbonyl (BOC), dimethoxytrityl (DMT) and triphenylmethyl (Trityl), even more preferably PG is tert-butyloxycarbonyl (BOC) or triphenylmethyl (Trityl).
25. The composition according to any one of items 1 to 19 for use in diagnostics.
26. The composition according to any one of items 1 to 19 for use in the imaging of Tau aggregates, particularly for use in positron emission tomography imaging of Tau aggregates.
27. A composition as defined in any one of items 1 to 19 for use in the diagnosis of a disorder associated with Tau aggregates or for use in the diagnosis of a tauopathy, particularly wherein the diagnosis is conducted by positron emission tomography.
28. A composition for use according to item 27, wherein the tauopathy is a 3R tauopathy.
29. A composition for use according to item 27, wherein the tauopathy is a 4R tauopathy.
30. The composition for use according to item 27, wherein the disorder is selected from Alzheimer's disease (AD), familial AD, Creutzfeldt-Jacob disease, dementia pugilistica, Down's Syndrome, Gerstmann-Striussler-Scheinker disease, inclusion-body myositis, prion protein cerebral amyloid angiopathy, traumatic brain injury (TBI), amyotrophic lateral sclerosis, Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease (PiD), progressive subcortical gliosis, progressive supranuclear palsy (PSP), subacute scerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, atypical parkinsonism, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, and lattice dystrophy.
31. The composition for use according to item 27, wherein the disorder is selected from Huntington's disease, ischemic stroke and psychosis in AD.
32. The composition for use according to item 30, wherein the disorder is Alzheimer's disease (AD).
33. The composition for use according to item 30, wherein the disorder is Parkinson's disease or atypical parkinsonism.
34. The composition for use according to item 30, wherein the disorder is progressive supranuclear paisy (PSP).
35. The composition for use according to item 30, wherein the disorder is Pick's disease (PiD).
36. The composition for use according to item 27, wherein the Tau aggregates are imaged in the brain or in the eye.
37. A method of imaging of Tau aggregates, particulary a method of positron emission tomography imaging of Tau aggregates, wherein an effective amount of a composition as defined in any one of items 1 to 19 is administered to a patient.
38. A method of diagnosing a disorder associated with Tau aggregates or a tauopathy, wherein an effective amount of a composition as defined in any one of items 1 to 19 is administered to a patient, particulary wherein the diagnosis is conducted by positron emission tomography.
39. A method according to item 38, wherein the tauopathy is a 3R tauopathy.
40. A method according to item 38, wherein the tauopathy is a 4R tauopathy.
41. The method according to item 38, wherein the disorder is selected from Alzheimer's disease (AD), familial AD, Creutzfeldt-Jacob disease, dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis, Parkinsonism-dementia complex of Guam, non-Guamanian motor neuron disease with neurofibrillary tangles, argyrophilic grain disease, corticobasal degeneration, diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease, progressive subcortical gliosis, progressive supranuclear palsy (PSP), subacute sclerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, atypical parkinsonism, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, and lattice dystrophy.
42. The method according to item 38, wherein the disorder is selected from Huntington's disease, ischemic stroke and psychosis in AD.
43. The method according to item 41, wherein the disorder is Alzheimer's disease (AD).
44. The method according to item 41, wherein the disorder is Parkinson's disease or atypical parkinsonism.
45. The method according to item 41, wherein the disorder is progressive supranuclear palsy (PSP).
46. The method according to item 41, wherein the disorder is Pick's disease (PiD).
47. The method according to item 41, wherein the Tau aggregates are imaged in the brain or in the eye.
48. Use of a composition as defined in any one of items 1 to 19 for the manufacture of an agent for imaging Tau aggregates, particulary for positron emission tomography imaging of Tau aggregates.
49. Use of a composition as defined in any one of items 1 to 19 for the manufacture of an agent for diagnosing a disorder associated with Tau aggregates or for diagnosing a tauopathy, particulary wherein the diagnosis is conducted by positron emission tomography.
50. The use according to item 49, wherein the tauopathy is a 3R tauopathy.
51. The use according to item 49, wherein the tauopathy is a 4R tauopathy.
52. The use according to item 49, wherein the disorder is selected from Alzheimer's disease (AD), familial AD, Creutzfeldt-Jacob disease, dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis, Parkinsonism-dementia complex of Guam, non-Guamanian motor neuron disease with neurofibrillary tangles, argyrophilic grain disease, corticobasal degeneration, diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease, progressive subcortical gliosis, progressive supranuclear palsy (PSP), subacute sclerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, atypical parkinsonism, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, and lattice dystrophy.
53. The use according to item 49, wherein the disorder is selected from Huntington's disease, ischemic stroke and psychosis in AD.
54. The use according to item 52, wherein the disorder is Alzheimer's disease (AD).
55. The use according to item 52, wherein the disorder is Parkinson's disease or atypical parkinsonism.
56. The use according to item 52, wherein the disorder is progressive supranuclear palsy (PSP).
57. The use according to item 52, wherein the disorder is Pick's disease (PiD).
58. The use according to item 49, wherein the Tau aggregates are imaged in the brain or in the eye.
59. Use of the composition according to any one of items 1 to 19 as an analytical reference.
60. Use of the composition according to any one of items 1 to 19 as an in vitro screening tool.
61. A method of collecting data for the diagnosis of a disorder associated with tau aggregates in a sample or a patient comprising:
   (a) bringing a sample or a specific body part or body area suspected to contain a tau aggregate into contact with a composition as defined in any one of items 1 to 19 which contains the compound of Formula I;
   (b) allowing the compound of Formula I to bind to the tau aggregate;
   (c) detecting the compound of Formula I bound to the tau aggregate; and
   (d) optionally correlating the presence or absence of compound of Formula I binding with the tau aggregate with the presence or absence of tau aggregate in the sample or specific body part or body area.
62. A method of determining the amount of tau aggregate in a tissue and/or a body fluid comprising:
   (a) providing a sample representative of the tissue and/or body fluid under investigation;
   (b) testing the sample for the presence of tau aggregate with a composition as defined in any one of items 1 to 19 which contains the compound of Formula I;
   (c) determining the amount of compound of Formula I bound to the tau aggregate; and
   (d) calculating the amount of tau aggregate in the tissue and/or body fluid.
63. A method of collecting data for determining a predisposition to a disorder associated with tau aggregates in a patient comprising detecting the specific binding of a composition as defined in any one of items 1 to 19, which contains the compound of Formula I, to a tau aggregate in a sample or in situ which comprises the steps of:
   (a) bringing the sample or a specific body part or body area suspected to contain the tau aggregate into contact with the composition as defined in any one of items 1 to 19, which contains compound of Formula I that specifically binds to the tau aggregate;
   (b) allowing the compound of Formula I to bind to the tau aggregate to form a compound/tau aggregate complex;
   (c) detecting the formation of the compound/tau aggregate complex;
   (d) optionally correlating the presence or absence of the compound/tau aggregate complex with the presence or absence of tau aggregate in the sample or specific body part or body area; and
   (e) optionaly comparing the amount of the compound/tau aggregate to a normal control value.
64. A method of collecting data for monitoring residual disorder in a patient suffering from a disorder associated with tau aggregates who has been treated with a medicament, wherein the method comprises:
   (a) bringing a sample or a specific body part or body area suspected to contain a tau aggregate into contact with a composition as defined in any one of items 1 to 19, which contains compound of Formula I that specifically binds to the tau aggregate;
   (b) allowing the compound of Formula I to bind to the tau aggregate to form a compound/tau aggregate complex;
   (c) detecting the formation of the compound/tau aggregate complex;
   (d) optionally correlating the presence or absence of the compound/tau aggregate complex with the presence or absence of tau aggregate in the sample or specific body part or body area; and
   (e) optionally comparing the amount of the compound/tau aggregate to a normal control value.
65. A method of collecting data for predicting responsiveness of a patient suffering from a disorder associated with tau aggregates and being treated with a medicament comprising:
   (a) bringing a sample or a specific body part or body area suspected to contain an tau aggregate into contact with a composition as defined in any one of items 1 to 19, which contains compound of Formula I that specifically binds to the tau aggregate;
   (b) allowing the compound of Formula I to bind to the tau aggregate to form a compound/tau aggregate complex;
   (c) detecting the formation of the compound/tau aggregate complex;
   (d) optionally correlating the presence or absence of the compound/tau aggregate complex with the presence or absence of tau aggregate in the sample or specific body part or body area; and
   (e) optionally comparing the amount of the compound/tau aggregate to a normal control value.

It is understood that the present invention covers compounds of the Formula I in which one or more of the respective atoms is replaced by a different isotope. For instance, the compounds of the Formula I include compounds in which one or more of the hydrogen atoms is replaced by tritium and/or one or more of the hydrogen atoms is replaced by deuterium.

### Definitions

The term “alkyl” refers to a saturated straight or branched carbon chain, which, unless specified otherwise, contain from 1 to 6 carbon atoms.

“Hal” or “halogen” represents F, Cl, Br and I. Preferably, “halogen” is, independently in each occurrence, selected from F, Cl and Br, more preferably, from F and Cl, even more preferably F.

The term “amine protecting group” (PG) as employed herein is any protecting group which is suitable for protecting an amine group during an envisaged chemical reaction. Examples of suitable protecting groups are well-known to a person skilled in the art. Suitable protecting groups are discussed, e.g., in the textbook Greene and Wuts, Protecting groups in Organic Synthesis, third edition, pages 494-653, which is included herein by reference. Protecting groups can be chosen from carbamates, amides, imides, N-alkyl amines, N-aryl amines, imines, enamines, boranes, N—P protecting groups, N-sufenyl, N-sulfonyl and N-silyl. Specific preferred examples of protecting groups (PG) are carbobenzyloxy (Cbz), (p-methoxybenzyl)oxycarbonyl (Moz or MeOZ), tert-butyloxycarbonyl (BOC), 9-fluorenylmethyloxycarbonyl (FMOC), benzyl (Bn), p-methoxybenzyl (PMB), 3,4-dimethoxybenzyl (DMPM), p-methoxyphenyl (PMP), triphenylmethyl (Trityl), methoxyphenyl diphenylmethyl (MMT), or dimethoxytrityl (DMT). More preferred examples of the protecting group PG include tert-butyloxycarbonyl (BOC), dimethoxytrityl (DMT) and triphenylmethyl (Trityl). One more preferred example of the protecting group PG is tert-butyloxycarbonyl (BOC).

The term “carbamate amine protecting group” refers to an amine protecting group containing a *—CO—O group wherein the asterisk indicates the bond to the amine. Examples are carbobenzyloxy (Cbz), (p-methoxybenzyl)oxycarbonyl (Moz or MeOZ), tert-butyloxycarbonyl (BOC) and 9-fluorenylmethyloxycarbonyl (FMOC).

The term “leaving group” (LG) as employed herein is any leaving group and means an atom or group of atoms can be replaced by another atom or group of atoms. Examples are given e.g. in Synthesis (1982), p. 85-125, table 2, Carey and Sundberg, Organische Synthese, (1995), page 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33). Preferably, the “leaving group” (LG) is selected from the group consisting of nitro, bromo, iodo, chloro, trialkyl ammonium, hydroxyl, boronic acid, iodonium, sulfonic ester. More preferably, the “leaving group” (LG) is nitro or trimethyl ammonium. It is to be understood that the compounds containing trialkyl ammonium or iodonium may further comprise an anion. Still more preferably, “leaving group” (LG) is nitro.

The term “crown ether” as employed herein means chemical compounds that consist of a ring containing several ether groups. More specifically, the term “crown ether” refers to preferably monocyclic organic groups which may be substituted and contain from 8 to 16 carbon atoms and from 4 to 8 heteroatoms selected from N, O and S in the ring. Each of the one or more optional substituents may be independently selected from any organic group containing from 1 to 15 carbon atoms and optionally 1 to 6 heteroatoms selected from N, O and S. Preferred examples of the “crown ether” are optionally substituted monocyclic rings containing 10 to 14 carbon atoms and 5 to 7 heteroatoms selected from N, O and S in the ring. Examples of the “crown ether” are optionally substituted monocyclic rings containing 12 carbon atoms and 6 heteroatoms selected from N and O in the ring. Specific examples include 18-crown-6, dibenzo-18-crown-6, and diaza-18-crown-6.

The term “cryptand” as employed herein relates to a class of polycyclic compounds related to the crown ethers, having three chains attached at two nitrogen atoms. A well-known “cryptand” is 4,7,13,16,21,24-hexaoxa-1,10-diazabicyclo[8.8.8]hexacosane (Kryptofix®).

Tau as used herein refers to a highly soluble microtubule binding protein mostly found in neurons and includes the major 6 isoforms, cleaved or truncated forms, and other modified forms such as arising from phosphorylation, glycosylation, glycation, prolyl isomerization, nitration, acetylation, polyamination, ubiquitination, sumoylation and oxidation. Pathologic Tau or Tau aggregates (Neurofibrillary Tangles, NFTs) as used herein refer to insoluble aggregates of the hyperphosphorylated Tau protein containing paired helical filaments and straight filaments. Their presence is a hallmark of AD and other diseases known as tauopathies.

The tau gene contains 16 exons with the major tau protein isoforms being encoded by 11 of them The alternative splicing of exon 10 generates tau isoforms with either three (exon 10 missing) or four (exon 10 present) repeat domains, known as 3R and 4R tau, respectively (A. Andreadis et al., Biochemistry 31, (1992) 10626-10633; M. Tolnay et al., IUBMB Life, 55(6): 299-305, 2003). In Alzheimer's disease, the ratio of 3R and 4R isoforms is similar. In contrast thereto, in some tauopathies one of the two isoforms is predominantly present. Herein, the term “3R tauopathy” refers to tauopathies (such as Pick's disease (PiD)) in which the 3R isoform is predominantly present. Herein, the term “4R tauopathy” refers to tauopathies (such as progressive supranuclear palsy (PSP) and corticobasal degeneration (CBD)) in which the 4R isoform is predominantly present.

As used hereinafter in the description of the invention and in the claims, the term “pharmaceutically acceptable salt” or “diagnostically acceptable salt” relates to non-toxic derivatives of the disclosed compounds wherein the parent compound is modified by making salts of inorganic and organic acids thereof. Inorganic acids include, but are not limited to, acids such as carboxylic, hydrochloric, nitric or sulfuric acid. Organic acids include, but are not limited to, acids such as aliphatic, cycoaliphatic, aromatic, araliphatic, heterocyclic, carboxylic and sulphonic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts can be found in Remington's Pharmaceutical Sciences, 18th ed., Mack Publishing Company, Easton, Pa., 1990, p. 1445, the disclosure of which is hereby incorporated by reference.

“Pharmaceutically acceptable” or “diagnostically acceptable” are defined as referring to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio. Preferably each of the components of the claimed compositions are pharmaceutically and diagnostically acceptable.

The patients or subjects in the present invention are typically animals, particularly mammals, more particularly humans.

“Chromatography” or “liquid chromatography” means a method for the separation of a mixture of compounds. The mixture is dissolved in a fluid and transported via “the mobile phase” through a “stationary phase”. The separation is based on the interaction of the compounds in the mobile phase with the stationary phases. Such different interactions result in differential retention on the stationary phase and thus affect the separation. Chromatography may be preparative or analytical. The purpose of preparative chromatography is to separate the components of a mixture, and is thus a form of purification. Analytical chromatography is done with a small sample of material and is used to measure the proportions of compounds in a mixture.

“High-performance liquid chromatography (HPLC)” is a form of liquid chromatography to separate compounds by using very small particles of the stationary phase (≤10 μm) and applying sufficiently higher pressures. An HPLC system typically consists of a reservoir of mobile phase(s), a pump, an injector, a separation column (containing the stationary phase), and detectors. For separation of radioactive compounds, suitable HPLC systems are equipped with a radioactivity detector. Optionally, the HPLC system has additional detectors, such as for example UV, photo diode array, refractive index, conductivity, fluorescence, mass spectrometer.

“Solid phase extraction (SPE)” is a sample preparation and/or purification process with two or more separate steps. First, the compounds are dissolved or suspended in a liquid mixture of solvents and the liquid sample is passed through a stationary (solid) phase. Some compounds are retained on the stationary phase while others pass through. In the second step, the retained compounds are eluted with a suitable solvent. Optionally, the stationary phase is washed with another solution before the elution step. In contrast to the HPLC technique, the used particle size is much bigger (e.g. ≥25 μm compared to HPLC with a typical particle size of s 10 μm) and therefore, the applied pressure is much lower (for HPLC the pressure is typically >50 bar).

“Solid phase extraction cartridge (SPE cartridge)” is a syringe or container (e.g. Sep Pak®) prefilled with the stationary phase for SPE.

“Sterile filtration” is a method for sterilization of a solution by filtration via a microfilter. A microfilter is a filter having, e.g., a pore size of about 0.25 μm or less, preferably about 20 nm to about 0.22 μm, which is usually used to remove microorganisms. Membrane filters used in microfiltration in production processes are commonly made from materiais such as mixed cellulose ester, polytetrafluorethylene (PTFE), polyvinylidene fluoride (PVDF) or polyethersulfone (PES).

“Automated” used herein, means the conduction of synthesis and or purification steps by a suitable apparatus (synthesizer).

The term “radioscavenger” refers to a compound that decreases the rate of decomposition due to radiolysis. Preferred radioscavengers include ascorbic acid and salts thereof and gentisic acid and salts thereof.

Suitable “synthesizers” for ¹³F-radiolabeling are well known to the person skilled in the art including but not limited to IBA Synthera, GE Fastlab, GE Tracerlab MX, GE Tracerlab FX, Trasis AllinOne, ORA Neptis Perform, ORA Neptis Mosaic, ORA Neptis Plug, Scintomics GPR, Synthera, Comecer Taddeo, Raytest Synchrom, Sofie Elixys, Eckert&Ziegler Modular Lab, Sumitomo Heavy Industries F100 F200 F300, Siemens Explora.

“Radiochemical purity” means that proportion of the total activity of the radionuclide present in its stated chemical form. Typically, the radiochemical purity is determined by thin-layer-chromatography or HPLC.

The term “hydroxycarboxylic acid” refers to a C₂-C₁₀ compound which has one or more carboxylic acid groups and one or more hydroxy groups (not including the hydroxy group(s) in the carboxylic acid group(s)). The hydroxycarboxylic acid can be saturated or unsaturated (including aromatic) and be cyclic or acyclic. In a preferred embodiment, the hydroxycarboxylic acid has one to three carboxylic acid groups. Preferably the hydroxycarboxylic acid has one to six hydroxy groups, more preferably one to four hydroxy groups. The hydroxycarboxylic acid can be in the form of the free acid or a cyclic ester thereof (i.e., lactone). Possible hydroxycarboxylic acids include, but are not limited to, ascorbic acid, hydroxybenzoic acids (such as gentisic acid), hydroxybenzoic acid derivatives, citric acid, lactic acid, malic acid, 2-hydroxybutanoic acid, 3-hydroxybutanoic acid, mandelic acid, gluconic acid, tartaric acid, and salicylic acid, preferably ascorbic acid, hydroxybenzoic acids (such as gentisic acid), hydroxybenzoic acid derivatives and citric acid.

The preferred definitions given in the “Definitions”-section apply to all of the embodiments described herein unless stated otherwise.

### DETAILED DESCRIPTION

In a first aspect, the invention is directed to a diagnostic composition comprising
a. a compound of Formula I,

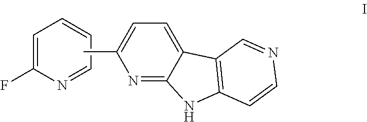

b. ethanol,
c. water, and
d. a hydroxycarboxylic acid, a salt of a hydroxycarboxylic acid or a mixture thereof.

F in Formula I is ¹⁸F or ¹⁹F. Preferably, F is ¹⁸F or a mixture of ¹⁸F and ¹⁹F.

Preferred compounds of the Formula I are selected from the group consisting of

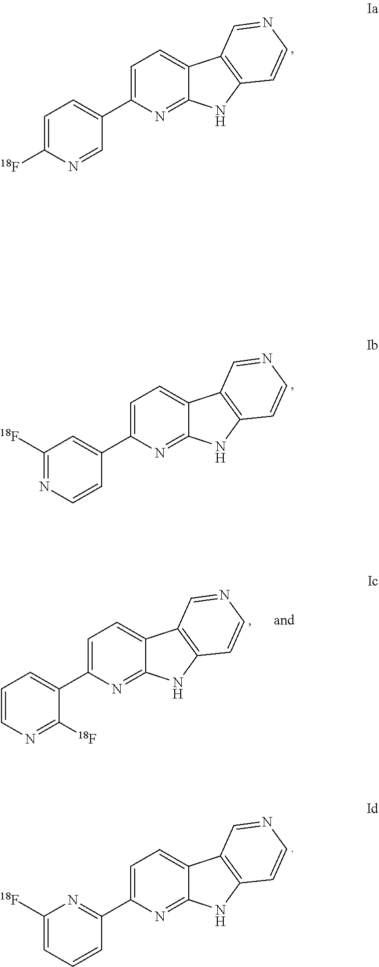 [SMILES: [18F]c1ccc(-c2ccc3c(n2)[nH]c2ccncc23)cn1 ; [18F]c1cc(-c2ccc3c(n2)[nH]c2ccncc23)ccn1 ; [18F]c1ncccc1-c1ccc2c(n1)[nH]c1ccncc12 ; [18F]c1cccc(-c2ccc3c(n2)[nH]c2ccncc23)n1]

A more preferred co pound of the Formula I is

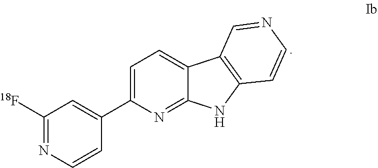 [SMILES: [18F]c1cc(-c2ccc3c(n2)[nH]c2ccncc23)ccn1]

Preferably, the diagnostic composition comprises about 0.03 GBq/mL to about 10 GBq/mL of the compound of Formula I. More preferably, the diagnostic composition comprises about 0.03 GBq/mL to about 5 GBq/mL of the compound of Formula I. Preferably, the diagnostic composition comprises at least about 1 GBq/mL of the compound of Formula I. More preferably, the diagnostic composition comprises at least about 2 GBq/mL of the compound of Formula I. Even more preferably, the diagnostic composition comprises at least about 3 GBq/mL of the compound of Formula I.

Preferably, the diagnostic composition comprises a maximum concentration of the compound of Formula I of about 10 μg/mL, more preferably a maximum concentration of the compound of Formula I of about 5 μg/mL.

Preferably, the diagnostic composition comprises about 1% v/v to about 20% v/v ethanol, based on the total amount of ethanol and water. More preferably, the diagnostic composition comprises about 1% v/v to about 15% v/v ethanol, based on the total amount of ethanol and water. Even more preferably, the diagnostic composition comprises about 5% v/v to about 10% v/v ethanol, based on the total amount of ethanol and water.

The diagnostic compositions comprise a hydroxycarboxylic acid, a salt of a hydroxycarboxylic acid or a mixture thereof. Any hydroxycarboxylic acid or a salt thereof can be employed. However, diagnostically acceptable hydroxycarboxylic acids or salts thereof are preferred. Preferably, the diagnostic composition comprises a hydroxycarboxylic acid, a salt of a hydroxycarboxylic acid or a mixture thereof, which is selected from the group consisting of ascorbic acid and ascorbic acid salts, hydroxybenzoic acids and salts of hydroxybenzoic acids, hydroxybenzoic acid derivatives and salts of hydroxybenzoic acid derivatives, citric acid and salts of citric acid and a mixture thereof. Preferably, the hydroxybenzoic acid derivatives are selected from the group comprising hydroxybenzoic acid, dihydroxybenzoic acid, and trihydroxybenzoic acid. More preferably, the dihydroxybenzoic acid derivative is gentisic acid.

More preferably, the diagnostic composition comprises one or more selected from ascorbic acid, sodium ascorbate, gentisic acid, gentisic acid sodium salt, citric acid, sodium citrate or a mixture thereof.

In one preferred embodiment, the diagnostic composition comprises about 2.5 to about 500 μmol/mL of a hydroxycarboxylic acid, a salt of a hydroxycarboxylic acid or a mixture thereof. More preferably, the diagnostic composition comprises about 10 to about 300 μmol/mL of a hydroxycarboxylic acid, a salt of a hydroxycarboxylic acid or a mixture thereof. Even more preferably, the diagnostic composition comprises about 25 to about 300 μmol/mL of a hydroxycarboxylic acid, a salt of an organic acid or a mixture thereof.

In another preferred embodiment, the diagnostic composition comprises ascorbic acid, sodium ascorbate or a mixture thereof (as the hydroxycarboxylic acid, the salt of the hydroxycarboxylic acid or the mixture thereof). Preferably, the diagnostic composition comprises about 10 to about 500 μmol/mL ascorbic acid, sodium ascorbate or a mixture thereof. More preferably, the diagnostic composition comprises about 50 to about 500 μmol/mL ascorbic acid, sodium ascorbate or a mixture thereof. Even more preferably, the diagnostic composition comprises about 100 to about 500 μmol/mL ascorbic acid, sodium ascorbate or a mixture thereof. The diagnostic composition may also comprise about 50 to about 300 μmol/mL ascorbic acid, sodium ascorbate or a mixture thereof. Still more preferably, the diagnostic composition comprises about 200 to about 300 μmol/mL ascorbic acid, sodium ascorbate or a mixture thereof.

In a further preferred embodiment, the diagnostic composition comprises gentisic acid, gentisic acid sodium salt or a mixture thereof (as the hydroxycarboxylic acid, the salt of the hydroxycarboxylic acid or the mixture thereof). Preferably, the diagnostic composition comprises about 2.5 to about 100 μmol/mL gentisic acid, gentisic acid sodium salt or a mixture thereof. More preferably, the diagnostic composition comprises about 10 to about 100 μmol/mL gentisic acid, gentisic acid sodium salt or a mixture thereof. Even more preferably, the diagnostic composition comprises about 25 to about 75 μmol/mL gentisic acid, gentisic acid sodium salt or a mixture thereof.

Preferably, the diagnostic composition comprises citric acid, sodium citrate or a mixture thereof (as the hydroxycarboxylic acid, the salt of the hydroxycarboxylic acid or the mixture thereof). Preferably, the diagnostic composition comprises about 10 to about 500 μmol/mL citric acid, sodium citrate or a mixture thereof. More preferably, the diagnostic composition comprises about 50 to about 500 μmol/mL citric acid, sodium citrate or a mixture thereof. Even more preferably, the diagnostic composition comprises about 50 to about 300 μmol/mL citric acid, sodium citrate or a mixture thereof.

The hydroxycarboxylic acid, the salt of the hydroxycarboxylic acid or a mixture thereof act as a scavenger to prevent radiolytic decomposition of the compound of Formula I. Further preferably, the hydroxycarboxylic acid, the salt of the hydroxycarboxylic acid or a mixture thereof are diagnostically acceptable.

Optionally, the diagnostic composition comprises an inorganic acid, an organic acid, a base, a salt or a mixture thereof, each of which is preferably diagnostically acceptable, wherein the organic acid, the salt or a mixture thereof is/are different from the hydroxycarboxylic acid, the salt of the hydroxycarboxylic acid or the mixture thereof. In one embodiment, the inorganic acid, the organic acid, the base, the salt or the mixture thereof is/are used during the synthesis or purification of the compound of Formula I. In another embodiment, the inorganic acid, the organic acid, the base, the salt or the mixture thereof is/are used for adjustment of pH and/or ionic strength of the diagnostic composition.

Examples of suitable inorganic or organic acids, bases and salts include sodium chloride, potassium chloride, monosodium phosphate, disodium phosphate, trisodium phosphate, monopotassium phosphate, dipotassium phosphate, tripotassium phosphate, hydrochloric acid, phosphoric acid, sodium hydroxide and potassium hydroxide.

In addition to the above components the diagnostic composition comprises water. The amount of water is chosen, so that the total amount of the composition is 100%.

The diagnostic composition has a pH of about 4 to about 8.5, preferably about 4.5 to about 8.

In a preferred embodiment, the diagnostic composition is sterile.

The diagnostic compositions of the present invention are suitable for parental administration to mammals for conducting PET imaging.

In a second aspect, the invention is directed to a method for obtaining a diagnostic composition of the present invention. In one embodiment, the method comprises the steps of
a) reacting a compound of Formula II with a ¹³F fluorinating agent,

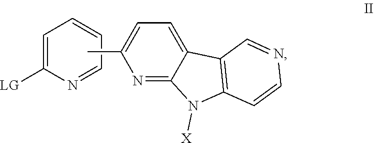

wherein X is H or PG,
LG is a leaving group, and
PG is an amine protecting group,
b) optionally, if X is PG, cleaving the protecting group PG,
c) purification of the compound of Formula I, and
d) optionally, mixing the compound of Formula I obtained in step c) with ethanol, water and a hydroxycarboxylic acid, a salt of a hydroxycarboxylic acid or a mixture thereof to provide a diagnostic composition.

Optionally sterile filtration (step e) can also be conducted.

The compound of Formula II is a precursor for the synthesis of a compound of Formula I.

Preferred compounds of the Formula II are selected from the group consisting of

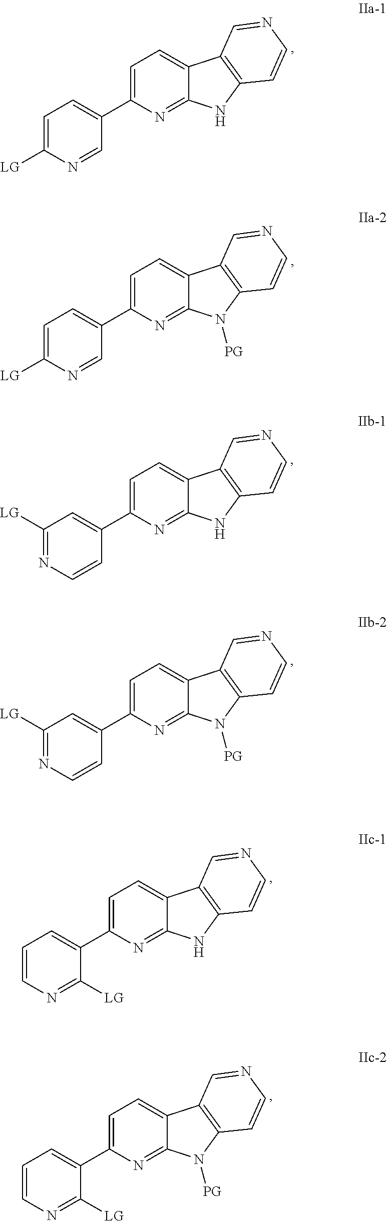

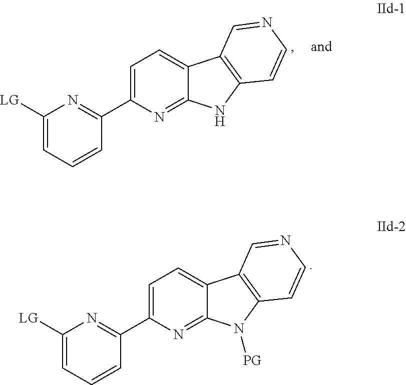

More preferred compounds of the Formula II are selected from the group consisting of

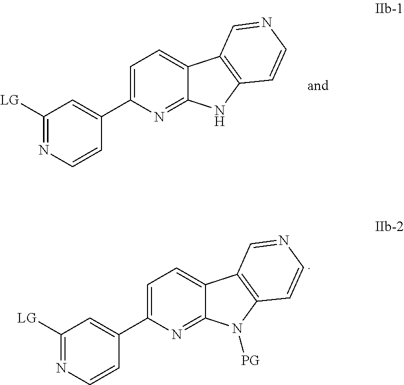

In these compounds PG and LG are as defined in the Definitions-section.

Even more preferred compounds of the Formula II are selected from the group consisting of

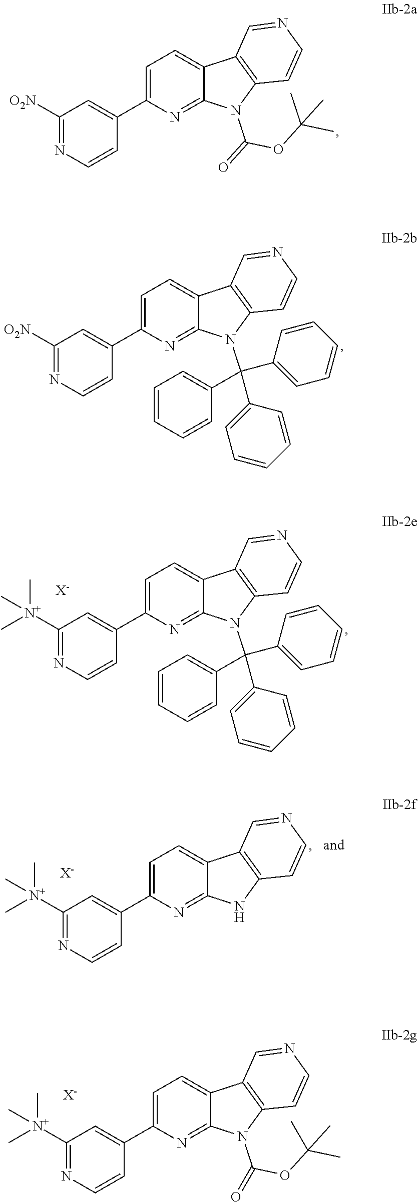

with X⁻ being a counter ion such as a counter ion selected from the group consisting of halogen, CF₃SO₃⁻, and CF₃CO₂⁻.

Still more preferred compounds of the Formula II are selected from the group consisting of

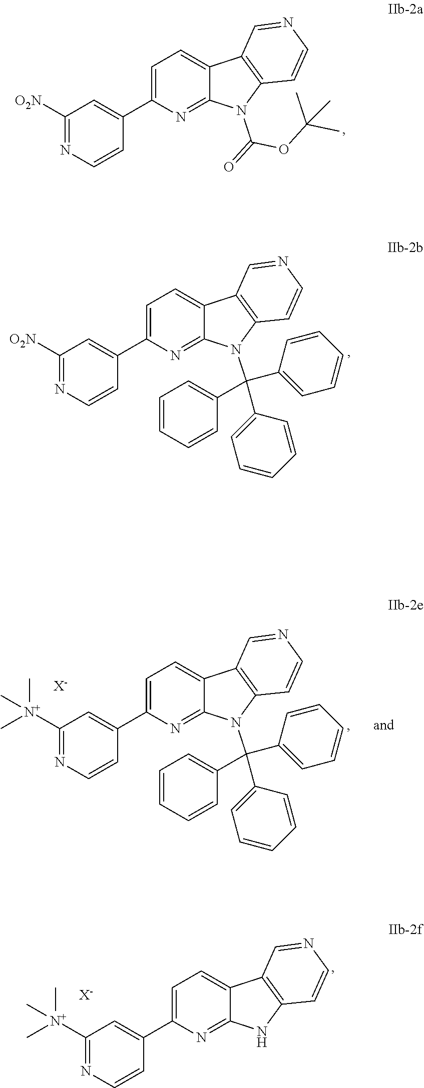

with X⁻ being a counter ion such as a counter ion selected from the group consisting of halogen, CF₃SO₃⁻, and CF₃CO₂⁻.

### Step a)

Step a) comprises reacting a compound of the Formula II with a ¹⁸F fluorinating agent

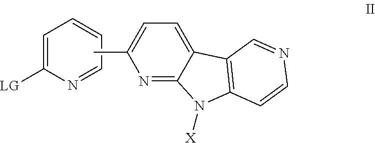

wherein

### X is H or PG,

LG is a leaving group, and
PG is an amine protecting group

If X is H a compound having the Formula I will result. If X is PG an intermediate compound having the Formula II will be obtained.

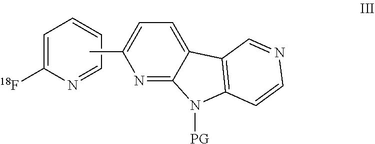

¹⁸F fluorinating agents are well known to the person skilled in the art. Any suitable ¹⁸F-fluorinating agent can be employed. Typical examples include H¹⁸F, alkali or alkaline earth ¹⁸F-fluorides (e.g., K¹⁸F, Rb¹⁸F, Cs¹⁸F, and Na¹⁸F). Optionally, the ¹⁸F-fluorinating agent can be used in combination with a chelating agent such as a cryptand (e.g.: 4,7,13,16,21,24-hexaoxa-1,10-diazabicyclo[8.8.8]-hexacosane-Kryptofix®) or a crown ether (e.g.: 18-crown-6). Alternatively, the ¹⁸F-fluorinating agent can be a tetraalkyl ammonium salt of ¹⁸F or a tetraalkyl phosphonium salt of ¹⁸F; e.g., tetra(C₁₋₆ alkyl)ammonium salt of ¹⁸F or a tetra(C₁₋₆ alkyl)phosphonium salt of ¹⁸F. Examples thereof include tetrabutyl ammonium [¹⁸F]fluoride and tetrabutyl phosphonium [¹⁸F]fluoride. Preferably, the ¹⁸F-fluorinating agent is K¹⁸F, H¹⁸F, Cs¹⁸F, Na¹⁸F or tetrabutyl ammonium [¹⁸F]fluoride. In an even more preferred embodiment, the ¹⁸F-fluorinating agent is K¹⁸F. In another more preferred embodiment, the ¹⁸F-fluorinating agent is tetrabutyl ammonium [¹⁸F]fluoride.

The ¹⁸F-fluorination is typically carried out in a solvent which is preferably selected from acetonitrile, dimethylsulfoxide, dimethytformamide, dimethylacetamide, amyl alcohol, ter-butyl alcohol, or a mixture thereof, preferably the solvent contains or is acetonitrile or DMSO. But also other solvents can be used which are well known to a person skilled in the art. The solvent may further comprise water and/or other alcohols, such as C₁₋₁₀ linear, branched or cyclic alkanols, as a co-solvent. In one preferred embodiment the solvent for carrying out the ¹⁸F radiolabeling contains dimethyl sulfoxide. In another preferred embodiment the solvent for carrying out the ¹⁸F radiolabeling contains acetonitrile. In one preferred embodiment the solvent for carrying out the ¹⁸F radiolabeling is dimethyl sulfoxide. In another preferred embodiment the solvent for carrying out the ¹⁸F radiolabeling is acetonitrile.

The ¹⁸F-fluorination is typically conducted for at most about 60 minutes. Preferred reaction times are at most about 30 minutes. Further preferred reaction times are at most about 15 minutes.

The ¹⁸F-fluorination is typically carried out at a temperature of about 60 to about 200° C. under conventional or microwave-supported heating. In a preferred embodiment, the ¹⁸F-fluorination is carried out at about 100 to about 180° C. In a more preferred embodiment, the ¹⁸F-fluorination is carried out at about 100 to about 160° C. Preferably, the F-fluorination is carried out under conventional heating. Conventional heating is understood to be any heating without the use of microwaves.

The amount of starting material is not particularly limited. For example, about 0.5 to about 50 μmol of a compound of the Formula II can be used for the production of the compound of the Formula I in one batch. In a preferred embodiment, about 2 to about 25 μmol of a compound of the Formula II are used. In a more preferred embodiment, about 2.5 to about 15 μmol of a compound of the Formula II are used. In one embodiment at least about 2 μmol of a compound of the Formula II are used. In a preferred embodiment, at least about 2.5 μmol of a compound of the Formula II are used. In a more preferred embodiment, at least about 3 μmol of a compound of the Formula II are used.

If X is PG an intermediate compound having the Formula II will be obtained. The protecting group PG can either be cleaved during the step a) or in an optional subsequent step b).

Preferred compounds of the Formula III are selected from the group comprising

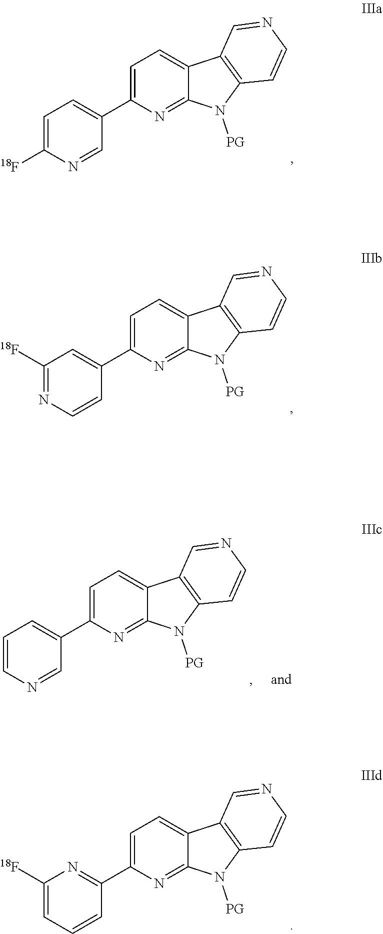

In these compounds PG is as defined in the “Definitions”-section.

### Step b)

Step b) is an optional step which comprises the cleavage of a protecting group PG from a compound of the Formula II to obtain a compound of the Formula I. As will be apparent to a skilled person, this step is not applicable if step a) is conducted with a compound of the Formula II in which X is hydrogen or if the protecting group PG is already cleaved in step a).

Reaction conditions for the cleavage of a large variety of protecting groups are well-known to a person skilled in the art and may be chosen from but are not limited to those described in the textbook by Greene and Wuts, Protecting groups in Organic Synthesis, third edition, page 494-653, and the textbook by P. J. Kocienski, Protecting Groups, 3rd Edition 2003, both of which are herewith included by reference.

The conditions which are employed in step b) will depend on the protecting group which is to be cleaved and are thus not particulary limited.

Possible reaction conditions include i) heating at about 60 to about 160° C., ii) addition of an acid and heating at about 0° C. to about 160° C.; or iii) addition of a base and heating at about 0° C. to about 160° C.

Preferred acids are hydrochloric acid, sulfuric acid, and phosphoric acid. One preferred acid is sulfuric acid. Another preferred acid is phosphoric acid. Preferred bases are sodium hydroxide, potassium hydroxide.

A preferred reaction condition is addition of an acid and heating at about 25° C. to 160° C., preferably 25° C. to 120° C.

If desired, Steps a) and b) can be performed in the same or different reaction vessels. Preferably, Steps a) and b) are performed in the same reaction vessel.

If desired, the solution obtained after Step b) can be used as such in Step c). Alternatively, the composition of the solution can be adapted, so that it is more appropriate for conducting HPLC. For instance, a buffer or diluent can be added prior to Step c).

### Step C)

Step c) comprises the purification of the compound of Formula I.

Suitable methods for purification of the compound of Formula I are HPLC, solid-phase-extraction (SPE) or a combination thereof.

In one preferred embodiment the compound of Formula I obtained in Step a) or, if employed, Step b), is subjected to HPLC using a mobile phase comprising ethanol and water and optionally an acid, a base, a buffer, a salt and/or a hydroxycarboxylic acid, a salt of a hydroxycarboxylic acid or mixture thereof.

The ratio of ethanol to water is not particularly limited but is preferably about 5/95 v/v to about 80/20 v/v, more preferably about 5/95 v/v to about 50/50 v/v, even more preferably about 5/95 v/v to about 20/80 v/v.

The pH of the mobile phase is not restricted, but it is preferably from about 0 to about 8, preferably about 0 to about 6, more preferably about 1 to about 5, even more preferably about 1 to about 3.

Possible buffers may include salts which can be selected from alkali metal dihydrogen phosphates, di alkali metal hydrogen phosphates, tri alkali metal phosphates, alkali metal acetates, alkali earth metal acetates, alkali earth metal formates, mono/di/tri alkali metal citrate, with the preferred alkali and alkali earth metals being sodium and potassium. Preferred buffers include salts which can be selected from alkali metal dihydrogen phosphates, dialkali metal hydrogen phosphates, trialkali metal phosphates, alkali metal acetates, mono/di/trialkali metal citrate, with the preferred alkali metals being sodium and potassium.

Possible bases can be sodium hydroxide and/or potassium hydroxide.

If desired, the pH of the mobile phase can be adjusted using an inorganic or organic acid.

Examples of inorganic acids include ascorbic acid, citric acid, and acetic acid. Examples of organic acids include hydrochloric acid, sulfuric acid, and phosphoric acid, preferably phosphoric acid.

A preferred mobile phase comprises about 5 to about 20% v/v ethanol, about 95 to about 80% v/v water, about 50 to about 150 mM buffer (e.g., alkali dihydrogen phosphate), with a pH of about 1 to about 3, and optionally a radioscavenger.

Stationary phases for use in HPLC methods are well-known and can be appropriately chosen by a skilled person. In a preferred embodiment, the stationary phase is a “reversed phase” (RP) stationary phase.

Examples of RP-HPLC stationary phases include C18, C8, phenyl, cyano (e.g. cyanopropyl), pentafluorphenyl, amino (e.g. aminopropyl), amide (e.g. C₁₀₋₂₄-alkanoic-aminopropyl), phenyl hexyl functionalized resins or mixed phase resins.

In one embodiment, the particle size of the HPLC stationary phase is about 1.6 to about 15 μm. In a preferred embodiment, the particle size of the HPLC stationary phase is about 5 to about 10 μm. In another embodiment, the particle size of the HPLC stationary phase is about 10 μm.

Typically, the HPLC column has a diameter of about 2.0 to about 50 mm and a length of about 50 to about 300 mm. In a preferred embodiment, the HPLC column has a diameter of about 4.6 to about 20 mm and a length of about 150 to about 250 mm. In a more preferred embodiment, the HPLC column has a dimension of 10×250 mm.

The flow rate employed in the high-performance liquid chromatography is not restricted and can be from about 1 to about 20 mL/min, more typically from about 2 to about 15 mL/min, even more typically from about 2 to about 7 mL/min.

The pressure employed in the high-performance liquid chromatography is not particularly limited and can be in the range of about 50 to about 400 bar, typically from about 50 to about 250 bar, more typically from about 50 to 200 bar.

Optional step d) comprises mixing the compound of Formula I obtained in step c) with one or more selected from the group consisting of ethanol, water, the hydroxycarboxylic acid and the salt of the hydroxycarboxylic acid, if they are not already present in the desired amount in admixture with the compound of Formula I after step c), to provide the diagnostic composition. Further optionally, one or more selected from an inorganic acid, a further organic acid, a base, or a salt may additionally be added in step d), if they are not already present in the desired amount in admixture with the compound of Formula I after step c).

If the diagnostic composition is to be administered to a patient it should be sterie. The diagnostic composition can be sterilized by any known method. One option is to conduct sterile filtration (step e). The sterile filter can be a standard sterie filter used for radiotracer filtration. Such sterile filters are well known in the art. Suitable sterile filters are polytetrafluoroethylene (PTFE) sterile filters (e.g. Millipore Millex-LG), polyethersulfone (PES) sterile filters (e.g. Millipore Millex-GP), polyvinylidene fluoride (PVDF) sterile filters (e.g. Millipore Millex-GV). More preferably, the hydrophobic filter is polytetrafluoroethylene (PTFE) sterile filter or polyvinylidene fluoride (PVDF) sterile filter.

Step e) can be performed after step d) or before step d), wherein the compound of Formula I obtained after step c) is subjected to sterie filteration and then optionally mixed with the other components of the diagnostic composition, wherein the other components of the pharmaceutical composition are sterile or are subjected to sterie filtration before mixing.

Preferably, Step a), Step b) and Step c) are performed by a synthesizer. More preferably, Step a), Step b), Step c) and Step d) are performed by a synthesizer. Even more preferably, Step a), Step b), Step c) Step d) and Step e) are performed by a synthesizer.

Examples of such suitable synthesis devices include, but are not limited, to IBA Synthera, GE Fastlab, GE Tracerlab MX, GE Tracerlab FX, Trasis AllinOne, ORA Neptis Perform, ORA Neptis Mosaic, ORA Neptis Plug, Scintomics GPR, Synthera, Comecer Taddeo, Raytest Synchrom, Sofie Elixys, Eckert&Ziegler Modular Lab, Sumitomo Heavy Industries F100 F200 F300, and Siemens Explore.

Preferably, Step a), Step b) and Step c) are performed remotely controlled. More preferably, Step a), Step b), Step c) and Step d) are performed remotely controlled. Even more preferably, Step a), Step b), Step c) Step d) and Step e) are performed remotely controlled. Preferably, Step a), Step b) and Step c) are automated. More preferably, Step a), Step b), Step c) and Step d) are automated. Even more preferably, Step a), Step b), Step c) Step d) and Step e) are automated.

### Diagnostic Procedures

The diagnostic composition of the present invention is preferably for use in diagnosis. In this case, F in the compound of Formula I is preferably ¹⁸F.

Accordingly, in a third aspect, the invention is directed to the diagnostic composition as defined in the first aspect for the use in diagnosis. The composition of the present invention is particularly suitable for imaging of Tau aggregates, e.g., by positron emission tomography (PET). It can be used in the diagnosis of a disorder (such as a neuropathological disorder) associated with Tau aggregates or in the diagnosis of a tauopathy, particularly if the diagnosis is conducted by positron emission tomography. The Tau aggregates can be in the human brain.

It has been found that the diagnostic compositions of the present invention are particularly suitable for imaging of Tau protein aggregates. With respect to Tau protein, the detectably labeled compounds of the Formula I are able to bind to various types of Tau aggregates such as pathologically aggregated Tau, hyperphosphorylated Tau, neurofibrillary tangles, paired helical filaments, straight filaments, neurotoxic soluble oligomers, polymers and fibrils.

Due to the above binding characteristics, the detectably labeled compounds of the Formula I are suitable for use in the diagnosis of disorders associated with Tau aggregates. The detectably labeled compounds of the Formula I are particularly suitable for positron emission tomography (PET) imaging of Tau deposits. Typically ¹⁸F labeled compounds of the Formula I are employed as detectably labeled compounds if the compounds are to be administered to a patient.

It is to be understood that, in the following examples, the detectably labeled compounds of the Formula I are preferably administered in the diagnostic composition of the present invention.

The diagnostic composition of the present invention can thus be used in a method for collecting data for the diagnosis of a disorder associated with tau aggregates in a sample or a patient, preferably a human, comprising:
(a) bringing a sample or a specific body part or body area suspected to contain a tau aggregate into contact with a composition which comprises the compound of Formula I;
(b) allowing the compound to bind to the tau aggregate;
(c) detecting the compound bound to the tau aggregate; and
(d) optionally correlating the presence or absence of compound binding with the tau aggregate with the presence or absence of tau aggregate in the sample or specific body part or body area.

A specific method for detection of Tau deposits in a subject (e.g., a human) may comprise the steps of:
1) administration of a suitable amount of the diagnostic composition to the subject,
2) optionally, waiting for distribution of the diagnostic composition in the subject,
3) conduction of position emission tomography (PET),
4) optionally, reconstruction of the PET imaging data, and
5) interpretation of the PET imaging data.

Preferably, the diagnostic composition is to be administered intravenously. The dose of the detectably labeled compounds of the formula I may vary depending on the exact compound to be administered, the weight of the subject, size and type of the sample, and other variables as would be apparent to a physician skilled in the art. Generally, volume of the diagnostic composition that is to be injected into a human subject can be about 0.1 to about 20 mL, preferably about 0.1 to about 10 mL, more preferably about 0.5 to about 10 mL. Preferably, about 100 to about 740 MBq of the diagnostic composition are to be administered, more preferably, about 100 to about 400 MBq, even more preferably about 150 to about 300 MBq.

Preferably, the PET image acquisition is performed for about 5 to about 30 min, preferably for about 5 to about 20 min, more preferably for about 10 to about 20 min. Preferably, the PET acquisition is started about 30 to about 120 min post injection of the diagnostic composition, more preferably about 30 to about 90 min post injection, even more preferably about 45 to about 60 min post injection. The interpretation of the PET imaging data is performed by visual assessment or by a quantification method.

In the imaging of Tau aggregates a detectably labeled compound of the Formula I is administered and the signal stemming from the compound that is specifically bound to the Tau aggregates is detected. The specific binding is a result of the high binding affinity of the compounds of the Formula I to the Tau aggregates.

In a preferred embodiment, a detectably labeled compound of the Formula I is employed for diagnosing whether a tauopathy (preferably Alzheimer's disease) is present. In this method a detectably labeled compound of the Formula I is administered to a patient who is suspected to suffer from a tauopathy (preferably Alzheimer's disease) or a sample obtained from such a patient and the signal stemming from the detectable label is detected, preferably by positron emission tomography (PET).

If no signal stemming from the detectable label is detected then the instant method can be used to exclude a tauopathy, which indicates that a neurological disorder other than a tauopathy is present.

In the methods of diagnosing a disorder associated with Tau protein aggregates such as Alzheimer's disease, or a predisposition therefor in a subject, the method comprising:
a) administering to the mammal a diagnostically effective amount of a detectably labeled compound of the Formula I;
b) allowing the detectably labeled compound of the Formula I to distribute into the tissue of interest (such as brain tissue or body fluids such as cerebrospinal fluid (CSF)); and
c) imaging the tissue of interest, wherein an increase in binding of the detectably labeled compound of the Formula I to the tissue of interest compared to a normal control level of binding indicates that the subject is suffering from or is at risk of developing a disorder associated with Tau protein aggregates.

The detectably labeled compounds of the Formula I can be used for imaging of Tau protein aggregates in any sample or a specific body part or body area of a patient which suspected to contain a Tau protein aggregate. The detectably labeled compounds of the Formula I are able to pass the blood-brain barrier. Consequently, they are particularly suitable for imaging of Tau protein aggregates in the brain, as well as in body fluids such as cerebrospinal fluid (CSF).

Diagnosis of a Tau disorder or of a predisposition to a Tau-associated disorder in a patient may be achieved by detecting the specific binding of a detectably labeled compound of the Formula I to the Tau protein aggregates in a sample or in situ, which includes:
(a) bringing the sample or a specific body part or body area suspected to contain the Tau protein aggregate into contact with a detectably labeled compound of the Formula I which binds the Tau protein aggregate;
(b) allowing the detectably labeled compound of the Formula I to bind to the Tau protein aggregate to form a compound/Tau protein aggregate complex (hereinafter “compound/Tau protein aggregate complex” will be abbreviated as “compound/protein aggregate complex”);
(c) detecting the formation of the compound/protein aggregate complex,
(d) optionally correlating the presence or absence of the compound/protein aggregate complex with the presence or absence of Tau protein aggregates in the sample or specific body part or area; and
(e) optionally comparing the amount of the compound/protein aggregate complex to a normal control value, wherein an increase in the amount of the compound/protein aggregate complex compared to a normal control value may indicate that the patient is suffering from or is at risk of developing a Tau-associated disorder.

After the sample or a specific body part or body area has been brought into contact with the detectably labeled compound of the Formula I, the compound is allowed to bind to the Tau protein aggregate. The amount of time required for binding will depend on the type of test (e.g., in vitro or in vivo) and can be determined by a person skilled in the field by routine experiments.

The compound which has bound to the Tau protein aggregate can be subsequently detected by any appropriate method. A preferred method is positron emission tomography (PET).

The presence or absence of the compound/protein aggregate complex is then optionally correlated with the presence or absence of Tau protein aggregates in the sample or specific body part or area. Finally, the amount of the compound/protein aggregate complex can be compared to a normal control value which has been determined in a sample or a specific body part or body area of a healthy subject, wherein an increase in the amount of the compound/protein aggregate complex compared to a normal control value may indicate that the patient is suffering from or is at risk of developing a Tau-associated disorder.

Predicting responsiveness of a patient suffering from a disorder associated with Tau protein aggregates and being treated with a medicament can be achieved by
(a) bringing a sample or a specific body part or body area suspected to contain a Tau protein aggregate into contact with a detectably labeled compound of the Formula I;
(b) allowing the detectably labeled compound of the Formula I to bind to the Tau protein aggregate to form a compound/protein aggregate complex;
(c) detecting the formation of the compound/protein aggregate complex;
(d) optionally correlating the presence or absence of the compound/protein aggregate complex with the presence or absence of Tau protein aggregate in the sample or specific body part or body area; and
(e) optionally comparing the amount of the compound/protein aggregate complex to a normal control value.

How steps (a) to (e) can be conducted has already been explained above.

In the method for predicting responsiveness the amount of the compound/protein aggregate complex can be optionally compared at various points of time during the treatment, for instance, before and after onset of the treatment or at various points of time after the onset of the treatment. A change, especially a decrease, in the amount of the compound/protein aggregate complex may indicate that the patient has a high potential of being responsive to the respective treatment.

The diagnostic composition of the present invention has a number of significant advantages:
it is chemically stable and can be stored at room temperature for at least 8 hours or even for at least 10 hours,
it is stable at concentrations of the compound of Formula I of up to 5 μg/mL, preferably of up to 10 μg/mL,
sterile filtration of the diagnostic composition can be conducted without significant loss of radioactivity,
it allows the administration to a subject without significant loss of radioactivity on syringes and other materials,
it has high purity and stability of the compound of Formula I at high radioactivity concentration, e.g. ≥2 GBq/mL, preferably ≥3 GBq/mL, more preferably ≥5 GBq/mL for 10 h, preferably for 12 h,
it allows high purity and stability of the compound of Formula I at high radioactivity levels per batch, e.g. ≥20 GBq, preferably ≥50 GBq, more preferably ≥100 GBq for 10 h, preferably for 12 h,
it can be used for detection of Tau deposits in subjects.

The present invention illustrated by the following examples which should not be construed as limiting.

### EXAMPLES

All reagents and solvents were obtained from commercial sources and used without further purification. Proton (¹H) spectra were recorded on a Bruker DRX-400 MHz NMR spectrometer or on a Bruker AV-400 MHz NMR spectrometer in deuterated solvents. Mass spectra (MS) were recorded on an Advion CMS mass spectrometer. Chromatography was performed using silica gel (Fluka: Silica gel 60, 0.063-0.2 mm) and suitable solvents as indicated in the specific examples. Flash purification was conducted with a Biotage Isolera One flash purification system using HP-Sil (Biotage) or puriFlash-columns (Interchim) and the solvent gradient indicated in the specific examples. Thin layer chromatography (TLC) was carried out on silica gel plates with UV detection.

### Abbreviations

| | |
|---|---|
| AD | Alzheimer's disease |
| BSA | bovine serum albumin |
| Boc, BOC | tert-butyloxycarbonyl |
| CBD | corticobasal degeneration |
| d.c. | corrected for decay |
| d | doublet |
| dd | doublet of doublet |
| ddd | doublet of doublet of doublet |
| dt | doublet of triplet |
| DMA | dimethylacetamide |
| DMF | N,N-dimethyl formamide |
| DMSO | dimethylsulfoxide |
| DMTrt-Cl | 4,4′-(chloro(phenyl)methylene)bis(methoxybenzene) |
| dppf | 1,1′-bis(diphenylphosphino)ferrocene |
| EI | electron ionisation |
| ELSD | evaporative light scattering detector |
| ESI | electrospray ionisation |
| EtOH | ethanol |
| FTD | Frontotemporal dementia |
| HPLC | high performance liquid chromatography |
| HC | Healthy control |
| GBq | Gigabequerel |
| K₂₂₂ | 4, 7, 13, 16, 21, 24-hexaoxa-1,10- |
| | diazabicyclo[8.8.8]-hexacosane (Kryptofix 222) |
| MBq | Megabequerel |
| MS | mass spectrometry |
| MeCN | acetonitrile |
| m | multiplet |
| mc | centered multiplet |
| n.c.a. | non-carrier-added |
| n.d.c. | not decay corrected |
| NMR | nuclear magnetic resonance spectroscopy : |
| | chemical shifts (δ) are given in ppm. |
| PBS | phosphate-buffered saline |
| PET | Positron-Emission-Tomography |
| PiD | Pick's disease |
| PSP | progressive supranuclear palsy |
| q | quadruplet (quartet) |
| RT | room temperature |
| s | singulet |
| t | triplet |
| Tau | Tau protein, Tau deposits, Tau aggregates |
| TBI | Traumatic brain injury |
| Trt | trityl (triphenylmethyl) |
| TLC | thin layer chromatography |
| % v/v | Percentage by volume |
| EOS | End of Synthesis |
| GBq | Giga Becquerel |
| MBq | Mega Becquerel |
| SPE | Solid-Phase-Extraction |
| WFI | Water for injection |

### Synthesis of Test Compounds

### Preparative Example A

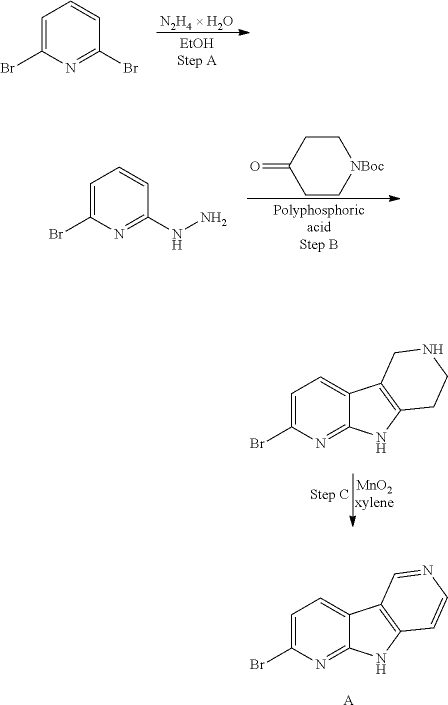

### Step A

Commercially available 2,6-dibromopyridine (4.12 g, 16.6 mmol) was suspended in ethanol (40 mL) and hydrazine hydrate (10 mL, 97.6 mmol) in water (˜50-60%) was added. The mixture was heated in a sand-bath at ˜115° C. for 18 hours. The solvent was removed and the residue was purified by chromatography on silica using ethyl acetate/n-heptane (60/40) to afford the title compound as an off-white solid (3.05 g, 93%).

¹H-NMR (400 MHz, CDCl₃): δ=7.33 (t, 1H), 6.83 (d, 1H), 6.67 (d, 1H), 6.00 (br-s, 1H), 3.33-3.00 (br-s, 2H)

### Step B

The title compound from Step A above (10 g, 53.2 mmol) and commercially available 1-Boc-4-piperidone (10.6 g, 53.2 mmol) were added to a 500 mL flask and mixed to become a homogenous blend. Then polyphosphoric acid (80 g, 115% H₃PO₄ basis) was added and the mixture was heated at ˜160° C. in a sand-bath. At ˜120° C. the Boc-protecting group was cleaved resulting in foaming of the reaction mixture. After complete Boc-cleavage the foam collapsed and the dark reaction mixture was stirred at ˜160° C. for 20 hours. The reaction mixture was allowed to cool to room temperature and water (400 mL) was added. The reaction mixture was stirred/sonicated until the gummy material was dissolved. The reaction mixture was then placed in an ice-bath and the pH of the solution was adjusted to pH˜12 by adding solid sodium hydroxide pellets (exothermic). The precipitate was collected by filtration and washed with water (400 mL) to remove salts. The precipitate was dissolved in dichloromethane/methanol (9/1; 1500 mL) by sonication and washed with water (2×400 mL) to remove remaining salts and insoluble material. The organic phase was dried over Na₂SO₄, filtered and the solvents were removed under reduced pressure. The dark residue was treated with dichloromethane (100 mL), sonicated for 5 minutes and the precipitate was collected by filtration. The precipitate was washed with dichloromethane (40 mL) and air-dried to afford the title compound as a beige solid (3.5 g, 26%).

¹H-NMR (400 MHz, DMSO-d₆): δ=11.5 (br-s, 1H), 7.72 (d, 1H), 7.15 (d, 1H), 3.86-3.82 (m, 2H), 3.06-3.00 (m, 2H), 2.71-2.65 (m, 2H)

### Step C

The title compound from Step B above (1.75 g, 6.94 mmol) was suspended in xylene (380 mL) and manganese (IV) oxide (6.62 g, 76.9 mmol) was added. The reaction mixture was then heated at ˜160° C. in a sand-bath for 36 hours. The cooled reaction mixture was evaporated under reduced pressure, the residue was suspended in dichloromethane/methanol (1/1; 400 mL) and stirred at room temperature for 30 minutes. The reaction mixture was then filtered through paper filters to remove the manganese (IV) oxide and the filter was washed with methanol (50 mL). The combined filtrates were evaporated under reduced pressure and the dark residue was purified by chromatography on silica (50 g HP-SIL-cartridge) using a Biotage Isolera system employing an ethyl acetate/heptane gradient (5/95-100/0) to remove unpolar impurities followed by dichloromethane/methanol (9/1->4/1) to afford the title compound as a dark yellow solid. The total yield from 2 runs was 1.77 g (51%).

¹H-NMR (400 MHz, DMSO-d₆): δ=12.52 (br-s, 1H), 9.42 (s, 1H), 8.61 (d, 1H), 8.53 (d, 1H), 7.56-7.52 (m, 2H)

### Preparative Example B

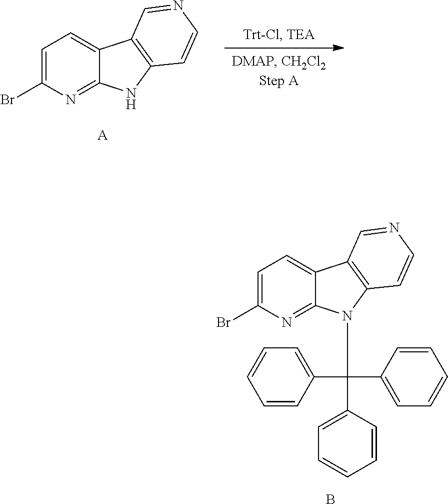

### Step A

To a suspension of the title compound from Preparative Example A (0.776 g, 0.72 mmol) in dichloromethane (65 mL) was added triethylamine (1.86 mL, 13 mmol) and trityl-chloride (2.63 g, 9.39 mmol). After the addition of 4-(dimethylamino)-pyridine (0.074 g, 0.608 mmol), the reaction mixture was stirred at room temperature for 16 hours. The reaction mixture was diluted with dichloromethane (150 mL) and water (50 mL). The organic phase was separated, dried over Na₂SO₄, filtered and the solvents were removed in vacuo. The residue was purified on HP-Sil SNAP cartridges (50 g) using a Biotage Isolera One purification system employing an ethyl acetate/n-heptane gradient (5/95->100/0->100/0) to afford the title compound B as a pale yellow solid (0.831 g, 54%). Unreacted starting material was recovered by flushing the cartridge with ethyl acetate/methanol (90/10) to afford the starting material as an off-white solid (0.195 g, 25%).

¹H-NMR (400 MHz, CDCl₃) δ=9.22 (s, 1H), 8.23 (d, 1H), 8.13 (d, 1H), 7.48-7.42 (m, 7H), 7.33-7.22 (m, 12H), 6.41 (d, 1H)

MS (ESI); m/z=490.03/491.96 [M+H]⁺

### Preparative Example C

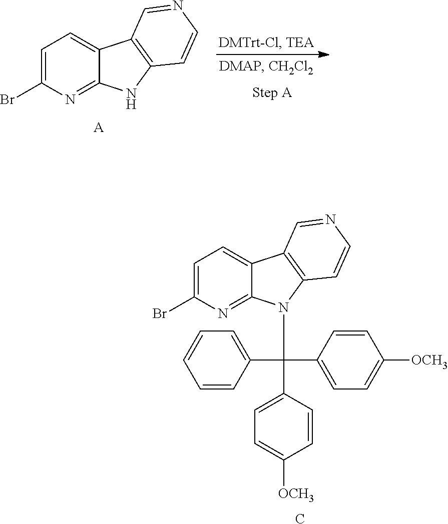

### Step A

To a suspension of the title compound from Preparative Example A (0.482 g, 1.94 mmol) in dichloromethane (40 mL) was added triethylamine (1.15 mL, 8 mmol) and 4,4′-(chloro(phenyl)methylene)bis(methoxybenzene; DMTrt-Cl) (1.963 g, 5.8 mmol). After the addition of 4-(dimethylamino)-pyridine (0.046 g, 0.377 mmol), the reaction mixture was stirred at room temperature for 3 days. The reaction mixture was diluted with dichloromethane (100 mL) and water (40 mL). The organic phase was separated, dried over Na₂SO₄, filtered and the solvents were removed in vacuo. The residue was purified on HP-Sil SNAP cartridges (50 g) using a Biotage Isolera One purification system employing an ethyl acetate/n-heptane gradient (5/95->100/0->100/0) to afford the title compound C as a pale yellow solid (0.825 g, 72%). Unreacted starting material was recovered by flushing the cartridge with ethyl acetate/methanol (90/10) to afford the starting material as an off-white solid (0.042 g, 8.8%).

¹H-NMR (400 MHz, CDCl₆) δ=9.23 (s, 1H), 8.23 (d, 1H), 8.13 (d, 1H), 7.39-7.31 (m, 6H), 7.29-7.25 (4H), 6.80 (d, 4H), 6.41 (dd, 1H), 3.81 (s, 6H)

### Example 1

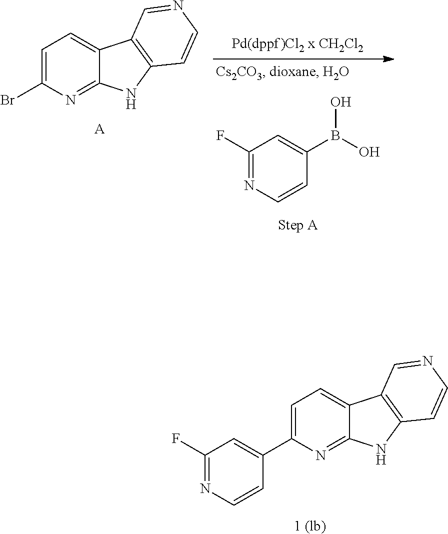

### Step A

To a mixture of degassed 1,4-dioxane (4.3 mL) and water (1 mL) in a microwave vial was added [1,1′-bis(diphenylphosphino)ferrocene]dichloropalladium(II), complex with dichloromethane (0.0084 g, 0.01 mmol), followed by the title compound of Preparative Example A (0.05 g, 0.2 mmol), (2-fluoropyridin-4-yl)boronic acid (0.035 g, 0.245 mmol) and cesium carbonate (0.133 g, 0.41 mmol). The reaction mixture was then heated at ˜115° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (25 g HP-SIL) using a Biotage Isolera system employing a dichoromethane/methanol gradient (100/0->95/5->90/10->80/20) to afford the title compound 1 (Ib) as an off-white solid (0.033 g, 63%).

¹H-NMR (400 MHz, DMSO-d₆) δ=12.50 (br-s, 1H), 9.45 (s, 1H), 8.83 (d, 1H), 8.56-8.52 (m, 1H), 8.43-8.39 (m, 1H), 8.19-8.14 (m, 2H), 7.92 (s, 1H), 7.54-7.50 (m, 1H)

MS (ESI): m/z=265.04 [M+H]⁺

### Example 2

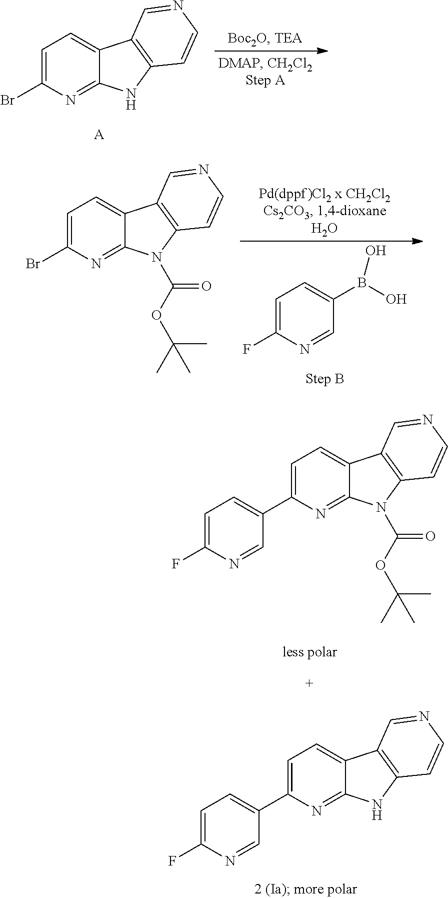

### Step A

To a suspension of the title compound of Preparative Example A (0.430 g, 1.73 mmol) in dichloromethane (25 mL) were added triethylamine (1.93 mL, 13.89 mmol) and di-tert-butyl dicarbonate (2.27 g, 10.02 mmol). After the addition of 4-(dimethylamino)-pyridine (0.042 g, 0.34 mmol), the reaction mixture was stirred at room temperature for 3 days. The solvents were removed under reduced pressure and the residue was purified on HP-Sil SNAP cartridges (25 g) using a Biotage Isolera One purification system employing an ethyl acetate/n-heptane gradient (5/95->100/0->100/0) to afford the title compound 2 (la) as an off-white solid (0.558 g, 92%).

¹H-NMR (400 MHz, CDCl₃) δ=9.28 (s, 1H), 8.73 (d, 1H), 8.22 (d, 2H), 7.59 8d, 1H), 1.80 (s, 9H)

### Step B

To a mixture of degassed 1,4-dioxane (3 mL) and water (0.7 mL) in a microwave vial was added [1,1′-bis(diphenylphosphino)ferrocene]dichloropalladium(II), complex with dichloro-methane (0.0058 g, 0.007 mmol), followed by the title compound from Step A above (0.05 g, 0.143 mmol), (6-fluoropyridin-3-yl)boronic acid (0.024 g, 0.17 mmol) and cesium carbonate (0.092 g, 0.286 mmol). The reaction mixture was then heated at ˜100° C. in a sand-bath for 4 hours. The reaction mixture was diluted with ethyl acetate (80 mL) and water (35 mL), the organic phase separated, dried over Na₂SO₄, filtered and the solvents were evaporated in vacuo. The dark residue was purified by chromatography on silica (12 g, puriFlash, Interchim) using a Biotage Isolera system employing a dichloromethane/methanol gradient (100/0->98/2->95/5->90/10->80/20) to afford the less polar Boc-protected compound (0.0255 g, 49%) and the more polar title compound 2 (la) as an off-white solid (0.0116 g, 31%).

More polar title compound 2 (la):

¹H-NMR (400 MHz, DMSO-d₆) δ=12.40 (br-s, 1H), 9.40 (s, 1H), 9.05 (s, 1H), 8.78-8.70 (m, 2H), 8.51 (d, 1H), 8.02 (d, 1H), 7.50 (d, 1H), 7.36 (dd, 1H)

MS (ESI): m/z=265.09 [M+H]⁺

Less polar Boc-protected compound:

¹H-NMR (400 MHz, DMSO-d₆) δ=9.48 (s, 1H), 9.13 (d, 1H), 8.84-8.78 (m, 2H), 8.68 (d, 1H), 8.23 (d, 1H), 8.19 (d, 1H), 7.40 (dd, 1H), 1.75 8s, 9H)

### Synthesis of Radiolabeling Precursors

### Example 3-A

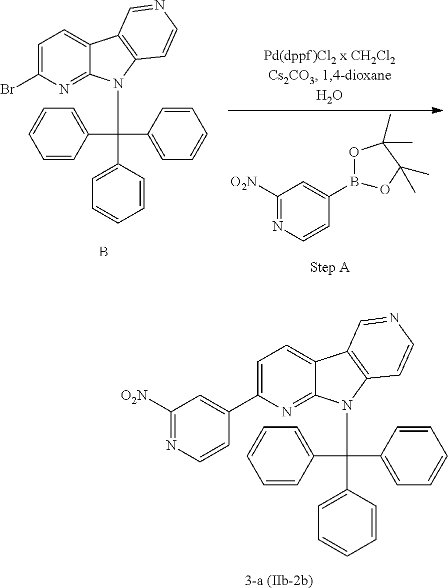

### Step A

To a mixture of degassed 1,4-dioxane (4.3 mL) and water (1 mL) in a microwave vial were added [1,1′-bis(diphenylphosphino)ferrocene]dichloropalladium(II), complex with dichloromethane (0.0084 g, 0.01 mmol), the title compound of Preparative Example B (0.1 g, 0.2 mmol), 2-nitro-4-(4,4,5,5-tetramethyl-1,3,2-dioxaborolan-2-yl)pyridine (0.061 g, 0.245 mmol) and cesium carbonate (0.133 g, 0.41 mmol). The reaction mixture was then heated at ˜115° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (25 g pufiFlash-column, Interchim) using a Biotage Isolera system employing an ethyl acetate/n-heptane gradient (5/95->100/0->100/0) to afford the title compound 3-a as a pale-yellow solid (0.082 g, 75%).

¹H NMR (400 MHz, CDCl₃) δ=9.32 (s, 1H); 8.56 (d, 1H), 8.48 (d, 1H), 8.33 (s, 1H); 8.30 (d, 1H), 7.85 (d, 1H), 7.69 (d, 1H), 7.58-7.54 (m, 5H), 7.32-7.25 (m, 10H), 6.48 (d, 1H)

MS (ESI): m/z=534.28 [M+H]⁺.

### Example 3-b

### Method a:

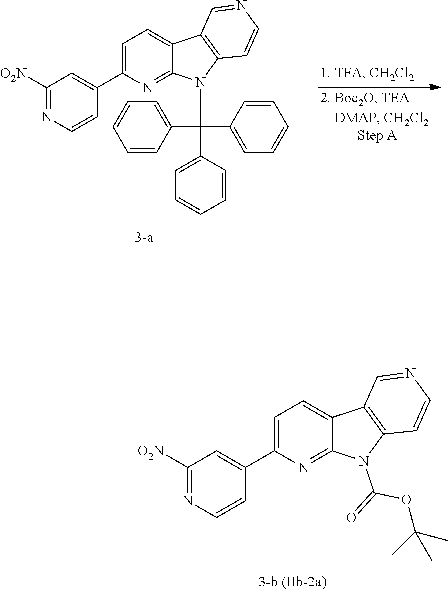 [SMILES: O=[N+]([O-])c1cc(-c2ccc3c4cnccc4n(C(c4ccccc4)(c4ccccc4)c4ccccc4)c3n2)ccn1 ; CC(C)(C)OC(=O)n1c2ccncc2c2ccc(-c3ccnc([N+](=O)[O-])c3)nc21]

### Step A

To a solution of 3-a (0.0396 g, 0.074 mmol) in dichloromethane (5 mL) was added trifluoroacetic acid (1.2 mL). The reaction mixture was stirred at room temperature for 6 hours and methanol (2 mL) was added. The solvents were evaporated in vacuo and the residue dissolved/suspended in methanol (5 mL). The solvents were evaporated in vacuo and the residue again dissolved/suspended in methanol (5 mL). The solvents were evaporated in vacuo and the residue suspended in dichloromethane (2 mL). After the addition of triethylamine (1 ml, 7.2 mmol), di-tert-butyl dicarbonate (0.098 g, 0.43 mmol), and 4-(dimethylamino)-pyridine (0.0018 g, 0.014 mmol), the reaction mixture was stirred at room temperature for 18 hours. The reaction mixture was diluted with ethyl acetate (50 mL) and water (20 mL). The organic phase was separated, dried over Na₂SO₄, filtered and the solvents were removed in vacuo. The residue was purified on silica (25 g puriFlash, Interchim) using a Biotage Isolera One purification system employing an ethyl acetate/n-heptane gradient (5/95->100/0->100/0) to elute unpolar byproducts followed by ethyl acetate/methanol (95/5) to afford the title compound 3-b pale as a yellow solid (0.0184 g, 63%).

¹H-NMR (400 MHz, CDCl₆) δ=9.36 (s, 1H), 9.15 (s, 1H), 8.82-8.76 (m, 2H), 8.57 (d, 1H), 8.45 (d, 1H), 8.36 (d, 1H), 8.07 (d, 1H), 1.87 (s, 9H)

MS (ESI); m/z=391.82 [M+H]⁺

### Method b:

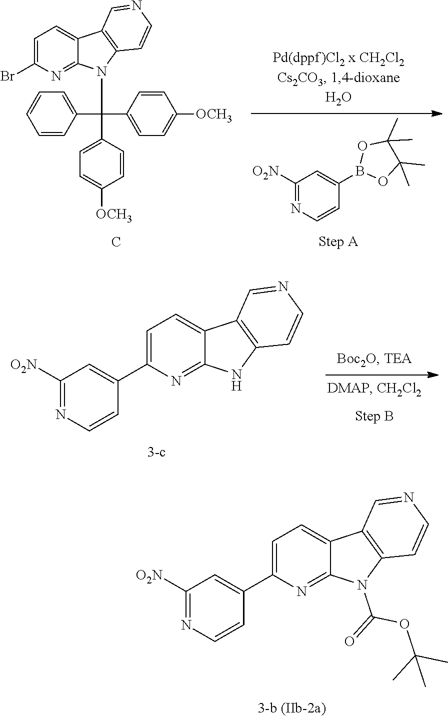

### Step A

To a mixture of degassed 1,4-dioxane (2.2 mL) and water (0.5 mL) in a microwave vial was added [1,1′-bis(diphenylphosphino)ferrocene]dichloropalladium(II), complex with dichloromethane (0.0042 g, 0.005 mmol), followed by the title compound of Preparative Example C (0.055 g, 0.1 mmol), 2-nitro-4-(4,4,5,5-tetrmethyl-1,3,2-dioxaborolan-2-yl)pyridine (0.0305 g, 0.12255 mmol) and cesium carbonate (0.067 g, 0.205 mmol). The reaction mixture was then heated at ˜115° C. in a sand-bath for 6 hours. The reaction mixture was diluted with ethyl acetate (20 mL), the precipitate collected by filtration, washed with water (10 mL) and methanol (5 mL) and air dried to afford 3-c (0.0277 g, 95%).

### Step B

To a suspension of the crude title compound from Step A above (0.0277 g, 0.095 mmol) in dichloromethane (4 mL) were added triethylamine (1 mL, 7.2 mmol), di-tert-butyl dicarbonate (0.2 g, 0.86 mmol), and 4-(dimethylamino)-pyridine (0.0036 g, 0.028 mmol). The reaction mixture was stirred at room temperature for 16 hours, diluted with ethyl acetate (50 mL) and water (20 mL). The organic phase was separated, dried over Na₂SO₄, filtered and the solvents were removed in vacuo. The residue was purified on silica (25 g puriFlash, Interchim) using a Biotage Isolera One purification system employing an ethyl acetate/n-heptane gradient (5/95->100/0->100/0) to elute unpolar byproducts followed by ethyl acetate/methanol (95/5) to afford the title compound 3-b as a pale yellow solid (0.0261 g, 70%).

¹H-NMR (400 MHz, CDCl₆s) δ=9.38 (s, 1H), 9.16 (s, 1H), 8.83-8.78 (m, 2H), 8.58 (d, 1H), 8.46 (d, 1H), 8.38 (d, 1H), 8.09 (d, 1H), 1.88 (s, 9H)

MS (ESI); m/z=391.85 [M+H]⁺: 291.74 [M+H-Boc]⁺

### Example 3-d

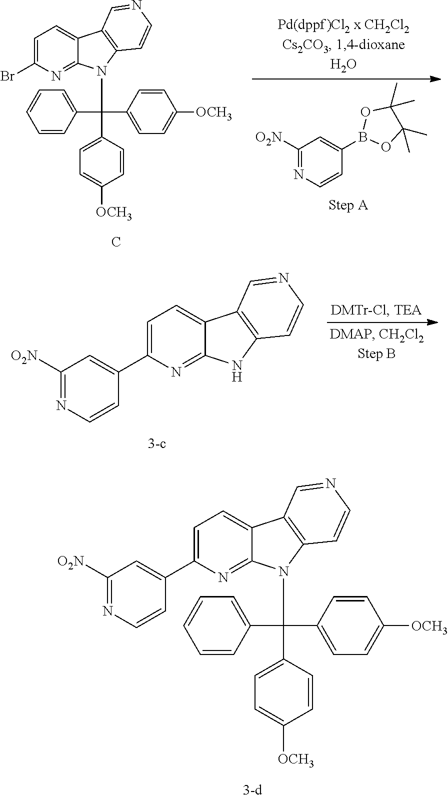

### Step A

To a mixture of degassed 1,4-dioxane (2.2 mL) and water (0.5 mL) in a microwave vial was added [1,1′-bis(diphenylphosphino)ferrocene]dichloropalladium(II), complex with dichloromethane (0.0042 g, 0.005 mmol), followed by the title compound of Preparative Example C (0.055 g, 0.1 mmol), 2-nitro-4-(4,4,5,5-tetramethyl-1,3,2-dioxaborolan-2-yl)pyridine (0.0305 g, 0.12255 mmol) and cesium carbonate (0.067 g, 0.205 mmol). The reaction mixture was then heated at ˜115° C. in a sand-bath for 6 hours. The reaction mixture was diluted with ethyl acetate (20 mL), the precipitate collected by filtration, washed with water (10 mL) and methanol (5 mL) and air dried to afford 3-c as a grey solid (0.0277 g, 95%).

### Step B

To a suspension of the crude title compound from Step A above (0.0277 g, 0.095 mmol) in dichloromethane (4 mL) were added triethylamine (1 mL, 7.2 mmol), 4,4′-(chloro(phenyl)methylene)bis(methoxybenzene) (0.081 g, 0.29 mmol), and 4-(dimethylamino)-pyridine (0.0036 g, 0.028 mmol). The reaction mixture was stirred at room temperature for 18 hours, diluted with ethyl acetate (50 mL) and water (20 mL). The organic phase was separated, dried over Na₂SO₄, filtered and the solvents were removed in vacuo. The residue was purified on silica (25 g puriFlash, Interchim) using a Biotage Isolera One purification system employing an ethyl acetate/n-heptane gradient (5/95->100/0->100/0) to afford the title compound 3-d as a pale yellow solid (0.0261 g, 44%).

¹H-NMR (400 MHz, CDCl₃) δ=9.32 (s, 1H), 8.58 (d, 1H), 8.50 (d, 1h), 8.36 (s, 1H), 8.30 (d, 1H), 7.85 (d, 1H), 7.74 (d, 1H), 7.52-7.42 (m, 6H), 7.27-7.23 (m, 4H), 6.80 (d, 4H), 6.49 (d, 1H), 3.78 (s, 6H)

### Example 3-9

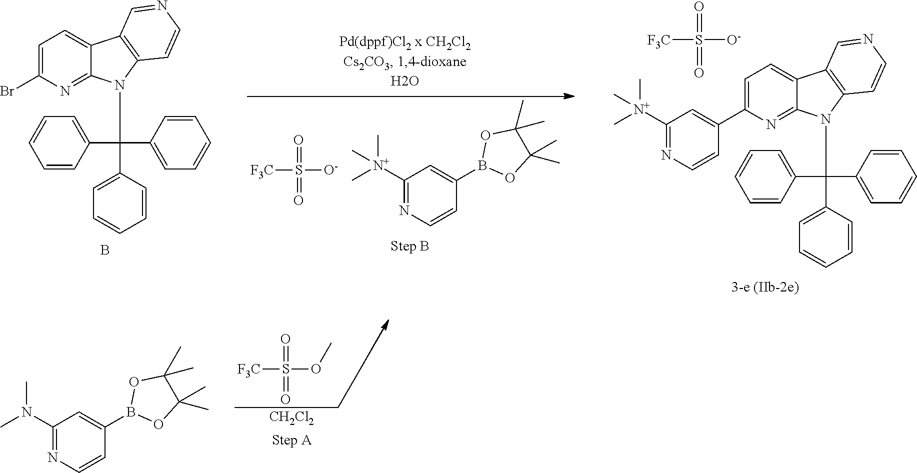

### Step A

Commercially available N,N-dimethyl-4-(4,4,5,5-tetramethyl-1,3,2-dioxaborolan-2-yl)pyridin-2-amine (0.25 g, 1 mmol) was dissolved in dichloromethane (5 mL). To the resultant stirring solution was added dropwise at room temperature methyl trifluoromethanesulfonate (0.124 mL, 1.1 mmol). The solution was stirred at room temperature for 4 hours. The reaction mixture was concentrated to remove dichloromethane and the residue was dried in vacuo to obtain a yellow glass/foam, which was directly used for the next step.

### Step B

To a solution of degassed 1,4-dioxane (12 mL) and water (3 mL) in a microwave vial were added [1,1′-bis(diphenylphosphino)ferrocene]dichloro-palladium(II), complex with dichloromethane (0.034 g, 0.04 mmol), the title compound of Preparative Example B (0.4 g, 0.816 mmol), the crude title compound from Step A above (˜1 mmol) and cesium carbonate (0.544 g, 1.68 mmol). The reaction mixture was heated at ˜120° C. in a sand-bath for 6 hours. The reaction mixture was diluted with ethyl acetate (150 mL) and water (50 mL), the organic phase separated, dried over Na₂SO₄, filtered and the solvents were evaporated in vacuo. The dark residue was purified by chromatography on silica (25 g HP-Ultra) using a Biotage Isolera system employing an ethyl acetate/n-heptane gradient (5/95->100/0->100/0) to elute unreacted starting material and unpolar byproducts. The gradient was then changed to dichloromethane/methanol (100/0->95/5->90/10) to afford the dimethylamine-derivative as pale yellow glass (0.127 g, 29%; MS (ESI): m/z=532.27 [M+H]+) and the methylamine-derivative as grey solid (0.0547 g, 13%; MS (ESI): m/z=519.18 [M+H]+). The gradient was again changed to dichloromethane/methanol (90/10->80/20) and held at (80/20) to obtain the title compound 3-e as a brown solid (0.104 g, 18%).

¹H NMR (400 MHz, DMSO-d6) δ=9.47 (s, 1H); 8.89 (d, 1H), 8.55 (d, 1H), 8-35-8.32 (m, 2H), 8.29 (d, 1H), 7.63-7.57 (m, 5H), 7.48 (d, 1H), 7.34-7.25 (m, 10H), 6.48 (d, 1H), 3.60 (s, 9H) MS (ESI): m/z=546.26 [M+H]⁺

### Example 3-f

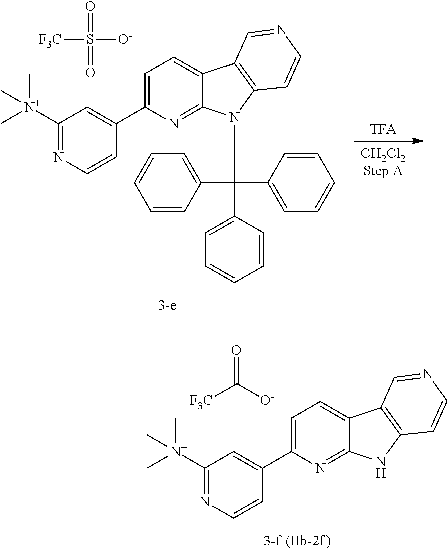 [SMILES: C[N+](C)(C)c1cc(-c2ccc3c4cnccc4n(C(c4ccccc4)(c4ccccc4)c4ccccc4)c3n2)ccn1 ; O=S(=O)([O-])C(F)(F)F ; "CH2Cl2Step A" ; "TFA" ; C[N+](C)(C)c1cc(-c2ccc3c(n2)[nH]c2ccncc23)ccn1 ; O=C([O-])C(F)(F)F]

### Step A

3-e (0.199 g, 0.364 mmol) was suspended in dichloromethane (10 mL). After the addition of trifluoro acetic acid (10 mL), the reaction mixture was stirred at room temperature for 18 hours. The solvents were removed under reduced pressure, the residue dissolved in methanol (10 mL) and the solvents removed under reduced pressure. The methanol treatment of the residue was repeated two more times. The residue was then suspended in dichloromethane (20 mL) and sonicated for ˜5 minutes. The precipitate was collected by filtration, washed with dichloromethane (10 mL) and air-dried to afford the title compound 3-f as a grey solid (0.127 g, 83%).

¹H NMR (400 MHz, DMSO-d6) δ=13.76 (br-s, 1H), 9.84 (s, 1H); 8.12 (d, 1H), 8.89 (d, 1H), 8.80 (d, 1H), 8.75 (s, 1H), 8.54-8.50 (m, 2H), 8.04 (d, 1H), 3.72 (s, 9H) MS (ESI): m/z=303.91 [M+H]⁺

### Example 4-a

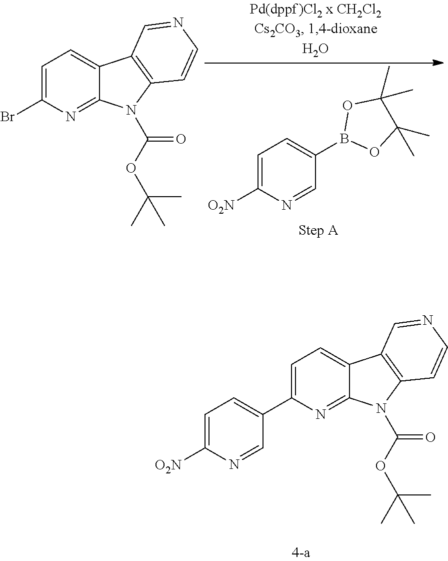 [SMILES: CC(C)(C)OC(=O)n1c2ccncc2c2ccc(Br)nc21 ; CC1(C)OB(c2ccc([N+](=O)[O-])nc2)OC1(C)C ; CC(C)(C)OC(=O)n1c2ccncc2c2ccc(-c3ccc([N+](=O)[O-])nc3)nc21]

### Step A

To a mixture of degassed 1,4-dioxane (3 mL) and water (0.7 mL) in a microwave vial was added [1,1′-bis(diphenylphosphino)ferrocene]dichloropalladium(II), complex with dichloro-methane (0.0058 g, 0.007 mmol), followed by the title compound from Example 2 Step A (0.05 g, 0.143 mmol), 2-nitro-5-(4,4,5,5-tetramethyl-1,3,2-dioxaborolan-2-yl)pyridine (0.0428 g, 0.17 mmol) and cesium carbonate (0.092 g, 0.286 mmol). The reaction mixture was then heated at ˜100° C. in a sand-bath for 4 hours. The reaction mixture was diluted with ethyl acetate (80 mL) and water (35 mL), the organic phase separated, dried over Na₂SO₄, filtered and the solvents were evaporated in vacuo. The dark residue was purified by chromatography on silica (12 g, puriFlash, Interchim) using a Biotage Isolera system employing a dichloromethane/methanol gradient (100/0->98/2->95/5->90/10->80/20) to afford the title compound 4-a as a pale yellow solid (0.0173 g, 31%).

¹H NMR (400 MHz, CDCl₃/CD₃D) δ=9.45 (d, 1H), 9.32 (s, 1H), 8.93 (dd, 1H), 8.68-8.64 (m, 2H), 8.46 (d, 1H), 8.35 (d, 1H), 8.14 (d, 1H), 1.82 (s, 9H)

MS (ESI): m/z=392.13 [M+H]⁺

### Radiolabeling of Precursors With ¹⁸F

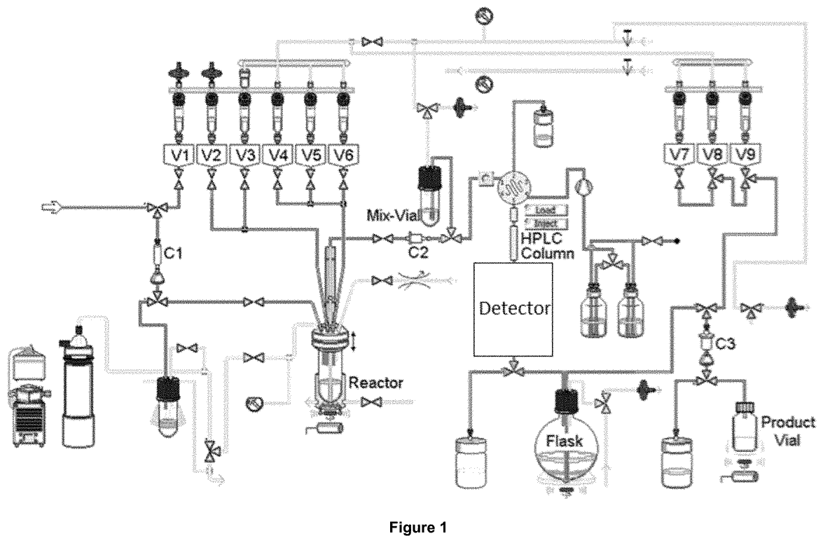

General radio labeling method A, performed on a tracerlab FX such as illustrated in FIG. 1 (Radiolabeling, deprotection, HPLC and SPE)—Comparative Examples

[¹³F]fluoride was trapped on a Sep-Pak Accell Plus QMA light cartridge (Waters) and eluted with a solution K₂CO₃/Kryptofix® 2.2.2. The water was removed using a stream of He or N₂ at 95° C. and co-evaporated to dryness with MeCN (1 mL). Afterwards, a solution of the dissolved precursor was added to the dried K[¹⁸F]F-Kryptofix complex. The reaction vial was sealed and heated for 15 min at 150° C. Subsequently, an acid (1-2 M HCl, 0.5-1M H₂SO₄ or 0.5-2M H₃PO₄) was added and the mixture was heated for 10 min at 150° C. The reaction mixture was diluted with 1 mL NaOH and 2.4 mL of the prep. HPLC mobile phase and the crude product was purified via semi-preparative HPLC (e.g. Phenomenex, Gemini C18, 5 μm, 250×10 mm) at 4 mL/min. The isolated tracer was diluted with water (20 mL+10 mg/mL sodium ascorbate), trapped on a C-18 Plus cartridge (Waters), washed with water (10 mL+10 mg/mL sodium ascorbate), eluted with ethanol (1 mL) and mixed with water (14 mL+10 mg/mL sodium ascorbate).

General radiolabeling method B. performed on a tracerlab FX such as illustrated in FIG. 1 (Radiolabeling, HPLC and SPE)—Comparative Examples

[¹⁸F]fluoride was trapped on a Sep-Pak Accell Plus QMA light cartridge (Waters) and eluted with a solution K₂CO₃/Kryptofix® 2.2.2. The water was removed using a stream of He or N₂ at 95° C. and co-evaporated to dryness with MeCN (1 mL). Afterwards, a solution of the dissolved precursor was added to the dried K[¹⁸F]F-Kryptofix complex. The reaction vial was sealed and heated for 15 min at 150° C. The reaction mixture was diluted with 0.5-1 mL NaOH and 2.4 mL of the prep. HPLC mobile phase and the crude product was purified via semi-preparative HPLC (e.g. Phenomenex, Gemini C18, 5 μm, 250×10 mm) at 4 mL/min. The isolated tracer was diluted with water (20 mL+10 mg/mL sodium ascorbate), trapped on a C-18 Plus cartridge (Waters), washed with water (10 mL+10 mg/mL sodium ascorbate), eluted with ethanol (1 mL) and mixed with water (14 mL+10 mg/mL sodium ascorbate).

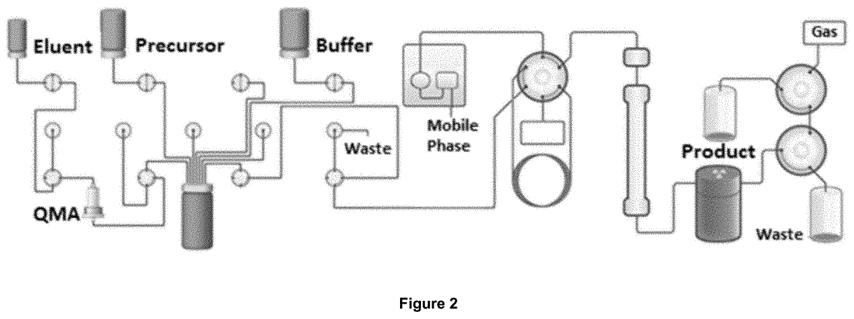

General radiolabeling method C performed on a IBA Synthera+Synthera HPLC such as illustrated in FIG. 2 (Radiolabeling, HPLC)

[¹³F]fluoride was trapped on a Sep-Pak Accell Plus QMA light cartridge (Waters) and eluted with a solution K₂CO₃/Kryptofix® 2.2.2. The water was removed using a stream of He or N₂ at 95-110° C. and co-evaporated to dryness. Afterwards, a solution of the dissolved precursor was added to the dried K[³F]F-Kryptofix complex. The reaction vial was sealed and heated for 15 min at 150° C. The reaction mixture was diluted with 0.5-1 mL 1M H₃PO₄ and 3-3.5 mL of the aqueous component of the prep. HPLC mobile phase and the crude product was purified via semi-preparative HPLC (e.g. Waters XBridge Peptide BEH C18, 130 Å, 10 μm, 10 mm×250 mm) at 3-6 mL/min. The fraction containing the product (5-10 mL) was collected and diluted with a dilution media containing 0-2 mL EtOH, 10-20 mL water, and 0-4 mL phosphate buffer concentrate (Braun, 3.05 g of disodium monohydrogen phosphate dodecahydrate, 0.462 g of sodium dihydrogen phosphate dihydrate in 20 mL of water for injection) and/or sodium ascorbate (100-1000 mg) and/or sodium citrate (100-1000 mg) and/or gentisic acid (20-200 mg).

General radiolabeling method D, performed on a tracerlab FX such as illustrated in FIG. 1 (Radiolabeling, HPLC)

[¹⁸F]fluoride was trapped on a Sep-Pak Accell Plus QMA light cartridge (Waters) and eluted with a solution K₂CO₃/Kryptofix® 2.2.2. The water was removed using a stream of He or N₂ at 95° C. and co-evaporated to dryness with MeCN (1 mL). Afterwards, a solution of the dissolved precursor was added to the dried K[¹⁸F]F-Kryptofix complex. The reaction vial was sealed and heated for 15 min at 150° C. The reaction mixture was diluted with 0.5-1 mL 1M H₃PO₄ and 3-3.5 mL of the aqueous component of the prep. HPLC mobile phase and the crude product was purified via semi-preparative HPLC (e.g. Waters XBridge Peptide BEH C18, 130 Å, 10 μm, 10 mm×250 mm or Gemini 5 μm C18, 250×10 mm, Phenomenex: 00G-4435-NO) at 3-6 mL/min. The fraction containing the product (5-10 mL) was collected and diluted with a dilution media containing 0-2 mL EtOH, 10-20 mL water, and 0-4 mL phosphate buffer concentrate (Braun) and/or sodium ascorbate (100-1000 mg) and/or sodium citrate (100-1000 mg) and/or gentisic acid (20-200 mg).

General radiolabeling method E. performed on a tracerlab FX such as illustrated in FIG. 1 (Radiolabeling, deprotection, HPLC)

[¹⁸F]fluoride was trapped on a Sep-Pak Accell Plus QMA light cartridge (Waters) and eluted with a solution K₂CO₃/Kryptofix® 2.2.2. The water was removed using a stream of He or N₂ at 95° C. and co-evaporated to dryness with MeCN (1 mL). Afterwards, a solution of the dissolved precursor was added to the dried K[¹⁸F]F-Kryptofix complex. The reaction vial was sealed and heated for 15 min at 150° C. Subsequently, 1 mL 0.5M H₂SO₄ was added and the mixture was heated for 10 min at 100° C. The reaction mixture was diluted with 0.5-1 mL 1M NaOH and 2-3 mL of the aqueous component of the prep. HPLC mobile phase and the crude product was purified via semi-preparative HPLC (e.g. Waters XBridge Peptide BEH C18, 130 Å, 10 μm, 10 mm×250 mm or Gemini 5 μm C18, 250×10 mm, Phenomenex: OOG-4435-NO) at 3-6 mL/min. The fraction containing the product (5-10 mL) was collected and diluted with a dilution media containing 0-2 mL EtOH, 10-20 mL water, and 0-4 mL phosphate buffer concentrate (Braun) and/or sodium ascorbate (100-1000 mg) and/or sodium citrate (100-1000 mg) and/or gentisic acid (20-200 mg).

Determination of the chemical and radiochemical Purity

Radiochemical and chemical purity was determined by analytical HPLC, e.g.: column: Atlantis T3, Waters, 100×4.6 mm, 3 μm, 100; mobile phase A: 40 mM sodium acetate, finally adjusted to pH 5.0 with glacial acetic acid; mobile phase B: 35% methanol in acetonitrile; flow rate: 1.8 mL/min; gradient: 0-5 min 15-32% B, 5-8 min 32-80% B, 8-12 min 80% B, 12-13 min 80-15% B, 13-16 min 15% B.

### Evaluation of Chemical Stability of Diagnostic Compositions

Mixtures according the composition described in Table 1 have been prepared. The chemical purity of compound 1(lb) was determined by HPLC (UV detection at 310 nm) after preparation of the composition as well as after storage at room temperature.

TABLE 1
Chemical Stability of compound 1 (Ib) in the diagnostic compositions

| | | | | Chem- |
|---|---|---|---|---|
| | | Chem- | | ical |
| | | ical | | purity |
| | | purity | Stor- | after |
| | Composition of the diagnostic | at base- | age | stor- |
| | composition | line | time | age |
| # | per 1 mL | [%] | [h] | [%] |
| 1 | 5 μg compound 1, 50 μL EtOH, 160 μL | 100 | 25 | 100 |
| | phosphate concentrate*, 586 μl WFI, | | | |
| | 204 μL 100 mM NaH₂PO₄ (acidified to pH | | | |
| | 2 with H₃PO₄) | | | |
| | Final pH: 6.9 | | | |
| 2 | 5 μg compound 1, 76 μL EtOH, 160 μL | 100 | 25 | 100 |
| | phosphate concentrate*, 560 μl WFI, | | | |
| | 204 μL 100 mM NaH₂PO₄ (acidified to pH | | | |
| | 2 with H₃PO₄) | | | |
| | Final pH: 6.9 | | | |
| 3 | 5 μg compound 1, 100 μL EtOH, 160 μL | 100 | 25 | 100 |
| | phosphate concentrate*, 536 μl WFI, | | | |
| | 204 μL 100 mM NaH₂PO₄ (acidified to pH | | | |
| | 2 with H₃PO₄) | | | |
| | Final pH: 7.0 | | | |
| 4 | 5 μg compound 1, 88 μL EtOH, 20 mg | 100 | 93 | 100 |
| | sodium citrate dihydrate, 602 μL WFI, | | | |
| | 272 μL 100 mM NaH₂PO₄ (acidified to pH | | | |
| | 2 with H₃PO₄), 4 μL conc. HCl, 34 μL 1 | | | |
| | M HCl | | | |
| | Final pH: 4.5 | | | |
| 5 | 5 μg compound 1, 88 μL EtOH, 20 mg | 100 | 93 | 100 |
| | sodium citrate dihydrate, 640 μL WFI, | | | |
| | 272 μL 100 mM NaH₂PO₄ (acidified to pH | | | |
| | 2 with H₃PO₄) | | | |
| | Final pH: 6.1 | | | |
| 6 | 5 μg compound 1, 88 μL EtOH, 20 mg | 100 | 92 | 100 |
| | sodium citrate dihydrate, 600 μL WFI, | | | |
| | 272 μL 100 mM NaH₂PO₄ (acidified to pH | | | |
| | 2 with H₃PO₄), 40 μL 1 M NaOH | | | |
| | Final pH: 7.1 | | | |
| 7 | 5 μg compound 1, 88 μL EtOH, 20 mg | 100 | 43 | 100 |
| | sodium citrate dihydrate, 638 μL WFI, | | | |
| | 272 μL 100 mM NaH₂PO₄ (acidified to pH | | | |
| | 2 with H₃PO₄), 1.6 μL conc. NaOH, 0.4 μL | | | |
| | 3 MNaOH | | | |
| | Final pH: 8.5 | | | |
| 8 | 5 μg compound 1, 88 μL EtOH, 2 mg | 100 | 20 | 100 |
| | gentisic acid, 476.4 μL WFI, 272 μL 100 | | | |
| | mM NaH₂PO₄ (acidified to pH 2 with | | | |
| | H₃PO₄), 160 μL phosphate concentrate*, | | | |
| | 2.8 μL H₃PO₄ 85%, 0.8 μL 3 M H₃PO₄ | | | |
| | Final pH: 4.5 | | | |
| 9 | 5 μg compound 1, 88 μL EtOH, 2 mg | 100 | 22 | 73.3 |
| | gentisic acid, 480 μL WFI, 272 μL 100 | | | |
| | mM NaH₂PO₄ (acidified to pH 2 with | | | |
| | H₃PO₄), 160 μL phosphate concentrate* | | | |
| | Final pH: 6.6 | | | |
| 10 | 5 μg compound 1, 88 μL EtOH, 2 mg | 40.9 | 19 | 18.6 |
| | gentisic acid, 450 μL WFI, 272 μL 100 | | | |
| | mM NaH₂PO₄ (acidified to pH 2 with | | | |
| | H₃PO₄), 160 μL phosphate concentrate*, | | | |
| | 30 μL M NaOH | | | |
| | Final pH: 8.5 | | | |
| 11 | 5 μg compound 1, 88 μL EtOH, 30 mg | 100 | 19 | 91.8 |
| | sodium ascorbate, 640 μL WFI, 272 μL | | | |
| | 100 mM NaH₂PO₄ (acidified to pH 2 with | | | |
| | H₃PO₄) | | | |
| | Final pH: 5.1 | | | |
| 12 | 5 μg compound 1, 88 μL EtOH, 50 mg | 100 | 17 | 96.6 |
| | sodium ascorbate, 640 μL WFI, 272 μL | | | |
| | 100 mM NaH₂PO₄ (acidified to pH 2 with | | | |
| | H₃PO₄) | | | |
| | Final pH: 5.5 | | | |

*phosphate buffer concentrate: Braun, 3.05 g of disodium monohydrogen phosphate dodecahydrate, 0.462 g of sodium dihydrogen phosphate dihydrate in 20 mL of water for injection

TABLE 2
Radiochemical Stability of compound ¹⁸F-1 (lb) in the diagnostic compositions
The compositions have been prepared according to the description in the general radiolabeling methods.

| | | General | | | | |
|---|---|---|---|---|---|---|
| | | radio- | | Radioactivity | Radiochemical | Radiochemical purity |
| | | labeling | | concentration at EOS | purity at EOS | after storage |
| # | Composition of the diagnostic composition | method | Precursor | [GBq/mL] | [%] | [%] |
| 1 | 36 GBq ¹⁸F-1, 1 mL EtOH, 14 mL sodium | A | 3-a | 3.3 | 97.6 | <10 (EOS + 4 h) |
| | ascorbate solution in water (10 mg/mL) | | | | | |
| 2 | 34 GBq ¹⁸F-1, 1.3 mL EtOH, 7.7 mL 70 mM | C | 3-b | 3.4 | 100 | <5 (EOS + 2 h) |
| | NaOAc (acidified to pH 2 with HCl), 1 mL | | | | | |
| | phosphate buffered saline (Gibco DPBS #: 14190), | | | | | |
| | 100 μl 5M NaOH | | | | | |
| 3 | 32 GBq ¹⁸F-1, 1.9 mL EtOH, 5.1 mL 100 mM | C | 3-b | 1.78 | 100 | 91.2 (EOS + 4 h) |
| | NaH₂PO₄ pH 2, 540 mg sodium ascorbate, 3.5 | | | | | 88.3% (EOS + 21 h) |
| | mL phosphate concentrate, 7.5 mL water | | | | | |
| 4 | 85 GBq ¹⁸F-1, 2.2 mL EtOH, 6.8 mL 100 mM | C | 3-b | 3.2 | 99.3 | 81.8 (EOS + 4 h) |
| | NaH₂PO₄ pH 2, 750 mg sodium ascorbate, 6 mg | | | | | |
| | ascorbic acid, 18 mL water | | | | | |
| 5 | 22 GBq ¹⁸F-1, 1.9 mL EtOH, 5.1 mL 100 mM | C | 3-b | 1.1 | 97.7 | 97.0 (EOS + 4 h) |
| | NaH₂PO₄ pH 2, 100 mg gentisic acid, 9.5 mL | | | | | |
| | water, 3.5 mL phosphate buffer concentrate* | | | | | |
| 6 | 102 GBq ¹⁸F-1, 2.2 mL EtOH, 6.8 mL 100 mM | C | 3-b | 3.8 | 99.7 | 98.7 (EOS + 6 h) |
| | NaH₂PO₄ pH 2, 100 mg gentisic acid, 13.5 mL | | | | | |
| | water, 4.5 mL phosphate buffer concentrate* | | | | | |
| 7 | 38 GBq ¹⁸F-1, 1.9 mL EtOH, 5.1 mL 100 mM | C | 3-b | 1.9 | 99.7 | 96.2 (EOS + 4 h) |
| | NaH₂PO₄ pH 2, 13 mL water, 500 mg sodium | | | | | 95.4 (EOS + 20 h) |
| | citrate | | | | | |
| 8 | 91 GBq ¹⁸F-1, 2.2 mL EtOH, 6.8 mL 100 mM | C | 3-b | 3.5 | 99.9 | >97 (EOS + 10 h) |
| | NaH₂PO₄ pH 2, 500 mg sodium citrate, 16 mL | | | | | |
| | water | | | | | |
| 9 | 114 GBq ¹⁸F-1, 2.2 mL EtOH, 6.8 mL 100 mM | C | 3-b | 4.4 | 99.8 | >91 (EOS + 8 h) |
| | NaH₂PO₄ pH 2, 17 mL water, 50 mg/mL sodium | | | | | |
| | ascorbate | | | | | |
| 9 | 107 GBq ¹⁸F-1, 2.2 mL EtOH, 6.8 mL 100 mM | C | 3-b | 4.1 | 99.5 | >92 (EOS + 10 h) |
| | NaH₂PO₄ pH 2, 17 mL water, 70 mg/mL sodium | | | | | |
| | ascorbate | | | | | |
| 10 | 105 GBq ¹⁸F-1, 2.2 mL EtOH, 6.8 mL 100 mM | C | 3-b | 4.0 | 99.7 | >93 (EOS + 8 h) |
| | NaH₂PO₄ pH 2, 17 mL water, 100 mg/mL sodium | | | | | |
| | ascorbate | | | | | |

*phosphate buffer concentrate: Braun, 3.05 g of disodium monohydrogen phosphate dodecahydrate, 0.462 g of sodium dihydrogen phosphate dihydrate in 20 mL of water for injection

TABLE 3
Filter retention
Mixtures according the composition described in Table 1 have been prepared. The filter
retention of compound 1 (Ib) was determined by comparing the corresponding peak area in
the analytical HPLC (UV detection at 310 nm) before and after filtration of 10 mL of the
diagnostic composition.

| | | % retention of compound 1 on sterile filter | | | | |
|---|---|---|---|---|---|---|
| | | | What- | | | |
| | | B. Braun, | man FP | Millex- | Millex | Millex- |
| | Composition of the diagnostic | Sterifix | (Cell. | GV | GP | LG |
| # | composition per 1 mL | (PES) | acetate) | (PVDF) | (PES) | (PTFE) |
| 1 | 5 μg compound 1, 50 μL EtOH, | 24.9 | 89.8 | 25.5 | 25.1 | 25.4 |
| | 160 μL phosphate concentrate*, | | | | | |
| | 586 μl WFI, 204 μL 100 mM | | | | | |
| | NaH₂PO₄ (acidified to pH 2 with | | | | | |
| | H₃PO₄) | | | | | |
| | Final pH: 6.9 | | | | | |
| 2 | 5 μg compound 1, 76 μL EtOH, | 5.7 | 82.6 | 5.4 | 4.3 | 4.0 |
| | 160 μL phosphate concentrate*, | | | | | |
| | 560 μl WFI, 204 μL 100 mM | | | | | |
| | NaH₂PO₄ (acidified to pH 2 with | | | | | |
| | H₃PO₄) | | | | | |
| | Final pH: 6.9 | | | | | |
| 3 | 5 μg compound 1, 100 μL EtOH, | 5.4 | 74.2 | 3.5 | 1.2 | 4.1 |
| | 160 μL phosphate concentrate*, | | | | | |
| | 536 μl WFI, 204 μL 100 mM | | | | | |
| | NaH₂PO₄ (acidified to pH 2 with | | | | | |
| | H₃PO₄) | | | | | |
| | Final pH: 7.0 | | | | | |
| 4 | 5 μg compound 1, 88 μL EtOH, 20 | <1 | 3.4 | 2.7 | <1 | <1 |
| | mg sodium citrate dihydrate, 602 μL | | | | | |
| | WFI, 272 μL 100 mM NaH₂PO₄ | | | | | |
| | (acidified to pH 2 with H₃PO₄), 4 μL | | | | | |
| | conc. HCl, 34 μL 1M HCl | | | | | |
| | Final pH: 4.5 | | | | | |
| 5 | 5 μg compound 1, 88 μL EtOH, 20 | <1 | 31.8 | 1.2 | <1 | <1 |
| | mg sodium citrate dihydrate, 640 μL | | | | | |
| | WFI, 272 μL 100 mM NaH₂PO₄ | | | | | |
| | (acidified to pH 2 with H₃PO₄) | | | | | |
| | Final pH: 6.1 | | | | | |
| 6 | 5 μg compound 1, 88 μL EtOH, 20 | 5.3 | 82.2 | 4.0 | 3.2 | 3.3 |
| | mg sodium citrate dihydrate, 600 μL | | | | | |
| | WFI, 272 μL 100 mM NaH₂PO₄ | | | | | |
| | (acidified to pH 2 with H₃PO₄), | | | | | |
| | 40 μL 1M NaOH | | | | | |
| | Final pH: 7.1 | | | | | |
| 7 | 5 μg compound 1, 88 μL EtOH, 20 | 8.8 | 98.2 | 7.4 | 6.8 | 11.3 |
| | mg sodium citrate dihydrate, 638 μL | | | | | |
| | WFI, 272 μL 100 mM NaH₂PO₄ | | | | | |
| | (acidified to pH 2 with H₃PO₄), | | | | | |
| | 1.6 μL conc. NaOH, 0.4 μL 3M | | | | | |
| | NaOH | | | | | |
| | Final pH: 8.5 | | | | | |
| 8 | 5 μg compound 1, 88 μL EtOH, 2 | <1 | 6.2 | 11.9 | — | <1 |
| | mg gentisic acid, 476.4 μL WFI, | | | | | |
| | 272 μL 100 mM NaH₂PO₄ (acidified | | | | | |
| | to pH 2 with H₃PO₄), 160 μL | | | | | |
| | phosphate concentrate*, 2.8 μL | | | | | |
| | H₃PO₄ 85%, 0.8 μL 3M H₃PO₄ | | | | | |
| | Final pH: 4.5 | | | | | |
| 9 | 5 μg compound 1, 88 μL EtOH, 2 | 2.6 | 48.8 | 4.5 | — | 4.0 |
| | mg gentisic acid, 480 μL WFI, | | | | | |
| | 272 μL 100 mM NaH₂PO₄ (acidified | | | | | |
| | to pH 2 with H₃PO₄), 160 μL | | | | | |
| | phosphate concentrate* | | | | | |
| | Final pH: 6.6 | | | | | |
| 10 | 5 μg compound 1, 88 μL EtOH, 2 | 9.3 | 100 | 12 | — | 13.8 |
| | mg gentisic acid, 450 μL WFI, | | | | | |
| | 272 μL 100 mM NaH₂PO₄ (acidified | | | | | |
| | to pH 2 with H₃PO₄), 160 μL | | | | | |
| | phosphate concentrate*, 30 μL 3M | | | | | |
| | NaOH | | | | | |
| | Final pH: 8.5 | | | | | |
| 11 | 5 μg compound 1, 88 μL EtOH, 30 | 1.5 | 8.1 | 1.9 | — | 2.8 |
| | mg sodium ascorbate, 640 μL WFI, | | | | | |
| | 272 μL 100 mM NaH₂PO₄ (acidified | | | | | |
| | to pH 2 with H₃PO₄) | | | | | |
| | Final pH: 5.1 | | | | | |
| 12 | 5 μg compound 1, 88 μL EtOH, 50 | <1 | 8.7 | <1 | — | <1 |
| | mg sodium ascorbate, 640 μL WFI, | | | | | |
| | 272 μL 100 mM NaH₂PO₄ (acidified | | | | | |
| | to pH 2 with H₃PO₄) | | | | | |
| | Final pH: 5.5 | | | | | |

*phosphate buffer concentrate: Braun, 3.05 g of disodium monohydrogen phosphate dodecahydrate, 0.462 g of sodium dihydrogen phosphate dihydrate in 20 mL of water for injection

1. A diagnostic composition comprising:
a. a compound of Formula I, 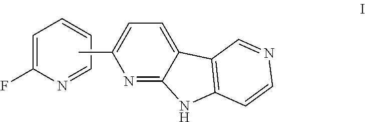
b. ethanol,
c. water, and
d. a hydroxycarboxylic acid, a salt of a hydroxycarboxylic acid or a mixture thereof.

2. A diagnostic composition according to claim 1, wherein F in Formula I is ¹⁸F or ¹⁹F, preferably ¹⁸F or a mixture of ¹⁸F and ¹⁹F.

3. A diagnostic composition according to claim 1, wherein the compound of Formula I is a compound of Formula Ib

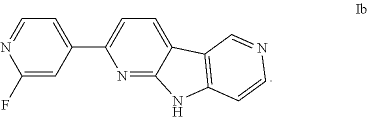 [SMILES: Fc1cc(-c2ccc3c(n2)[nH]c2ccncc23)ccn1]

4.-5. (canceled)

6. A diagnostic composition according to claim 1, wherein the hydroxycarboxylic acid, the salt of the hydroxycarboxylic acid or the mixture thereof are selected from the group consisting of ascorbic acid and salts of ascorbic acid, hydroxybenzoic acids and salts of hydroxybenzoic acids, hydroxybenzoic acid derivatives and salts of hydroxybenzoic acid derivatives, citric acid and salts of citric acid and a mixture thereof, preferably wherein the hydroxybenzoic acid derivative is selected from the group consisting of hydroxybenzoic acid, dihydroxybenzoic acid and trihydroxybenzoic acid, more preferably wherein the dihydroxybenzoic acid is gentisic acid.

7.-9. (canceled)

10. A diagnostic composition according to claim 1 comprising about 2.5 to about 500 μmol/mL of the hydroxycarboxylic acid, the salt of the hydroxycarboxylic acid or the mixture thereof, preferably about 10 to about 300 μmol/mL of the hydroxycarboxylic acid, the salt of the hydroxycarboxylic acid or the mixture thereof, more preferably about 25 to about 300 μmol/mL of the hydroxycarboxylic acid, the salt of the hydroxycarboxylic acid or the mixture thereof.

11.-13. (canceled)

14. A diagnostic composition according to claim 1 further comprising one or more of an inorganic acid, an organic acid, a base, or a salt, wherein the organic acid, the salt or the mixture thereof is/are different from the hydroxycarboxylic acid, the salt of the hydroxycarboxylic acid or the mixture thereof, preferably wherein the inorganic acid, the organic acid, the base, the salt or the mixture thereof is/are selected from the group consisting of sodium chloride, potassium chloride, monosodium phosphate, disodium phosphate, trisodium phosphate, monopotassium phosphate, dipotassium phosphate, tripotassium phosphate, hydrochloric acid, phosphoric acid, sodium hydroxide and potassium hydroxide.

15.-18. (canceled)

19. A method for manufacturing a diagnostic composition as defined in claim 1 comprising the steps of:
a. reacting a compound of Formula II with a ¹⁸F fluorinating agent, 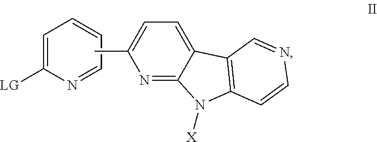
wherein X is H or PG,
LG is a leaving group, and
PG is an amine protecting group,
b. optionally, if X is PG, cleaving the protecting group PG,
c. purification of the compound of Formula I, and
d. optionally, mixing the compound of Formula I obtained in step c) with one or more selected from the group consisting of ethanol, water, the hydroxycarboxylic acidand the salt of the hydroxycarboxylic acid to provide the diagnostic composition, and
e. optionally sterile filtration before or after step d).

20.-21. (canceled)

22. A method according to claim 19, wherein LG in Formula II is a leaving group, which can be substituted by a nucleophilic [¹⁸F]fluoride ion or an electrophilic [¹⁸F]fluorine atom, preferably LG is selected from the group consisting of nitro, bromo, iodo, chloro, trialkyl ammonium, hydroxyl, boronic acid, iodonium, sulfonic ester, more preferably LG is nitro or trimethyl ammonium, wherein the compounds containing trialkyl ammonium or iodonium may further comprise an anion.

23. A method according to claim 19, wherein PG in Formula II is a protecting group, preferably PG is selected from the group consisting of carbobenzyloxy (Cbz), (p-methoxybenzyl)oxycarbonyl (Moz or MeOZ), tert-butyloxycarbonyl (BOC), 9-fluorenylmethyloxycarbonyl (FMOC), benzyl (Bn), p-methoxybenzyl (PMB), 3,4-dimethoxybenzyl (DMPM), p-methoxyphenyl (PMP), triphenylmethyl (Trityl), methoxyphenyl diphenylmethyl (MMT), or dimethoxytrityl (DMT), more preferably PG is selected from tert-butyloxycarbonyl (BOC), dimethoxytrityl (DMT) and triphenylmethyl (Trityl), even more preferably PG is tert-butyloxycarbonyl (BOC) or triphenylmethyl (Trityl).

24.-34. (canceled)

35. A method of imaging of Tau aggregates, particularly a method of positron emission tomography imaging of Tau aggregates, or a method of diagnosing a disorder associated with Tau aggregates of a tauopathy, wherein an effective amount of a composition as defined in claim 1 is administered to a patient, particularly wherein the diagnosis is conducted by a positron emission tomography.

36.-38. (canceled)

39. The method according to claim 35, wherein the disorder is selected from Alzheimer's disease (AD), familial AD, Creutzfeldt-Jacob disease, dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis, Parkinsonism-dementia complex of Guam, non-Guamanian motor neuron disease with neurofibrillary tangles, argyrophilic grain disease, corticobasal degeneration, diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease, progressive subcortical gliosis, progressive supranuclear palsy (PSP), subacute sclerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, atypical parkinsonism, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, lattice dystrophy, Huntington's disease, ischemic stroke and psychosis in AD.

40. The method according to claim 39, wherein the disorder is Alzheimer's disease (AD), Parkinson's disease, atypical parkinsonism, progressive supranuclear palsy (PSP), or Pick's disease (PiD).

41.-54. (canceled)

55. An analytical reference comprising the composition according to claim 1.

56. An in vitro screening tool comprising the composition according to claim 1.

57. (canceled)

58. A method of determining the amount of tau aggregate in a tissue and/or a body fluid comprising:
(a) providing a sample representative of the tissue and/or body fluid under investigation;
(b) testing the sample for the presence of tau aggregate with a composition as defined in claim 1 which contains the compound of Formula I;
(c) determining the amount of compound of Formula I bound to the tau aggregate; and
(d) calculating the amount of tau aggregate in the tissue and/or body fluid.

59. A method which comprises the steps of:
(a) bringing the sample or a specific body part or body area suspected to contain the tau aggregate into contact with the composition as defined in claim 1, which contains compound of Formula I that specifically binds to the tau aggregate;
(b) allowing the compound of Formula I to bind to the tau aggregate to form a compound/tau aggregate complex;
(c) detecting the formation of the compound/tau aggregate complex;
(d) optionally correlating the presence or absence of the compound/tau aggregate complex with the presence or absence of tau aggregate in the sample or specific body part or body area; and
(e) optionally comparing the amount of the compound/tau aggregate to a normal control value, wherein the method is
a method of collecting data for determining a predisposition to a disorder associated with tau aggregates in a patient comprising detecting the specific binding of a composition as defined in claim 1, which contains the compound of Formula I, to a tau aggregate in a sample of in situ,
a method of collecting data for monitoring residual disorder in a patient suffering from a disorder associated with tau aggregates who has been treated with a medicament, or
a method of collecting data for predicting responsiveness of a patient suffering from a disorder associated with tau aggregates and being treated with a medicament.

60.-61. (canceled)
